(19) United States
(12) Patent Application Publication
(10) Pub. No.: US 2014/0341893 A1
(43) Pub. Date: Nov. 20, 2014

(54) BLOOD PLASMA BIOMARKERS FOR BEVACIZUMAB COMBINATION THERAPIES FOR TREATMENT OF BREAST CANCER

(71) Applicant: Genentech, Inc., South San Francisco, CA (US)

(72) Inventors: Herbert Andres, Penzberg (DE); Sanne Lysbet de Haas, Basel (CH); Rebecca Elliott, San Francisco, CA (US); Johann Karl, Peissenberg (DE); Yu-Ju Gloria Meng, Albany, CA (US); Gregory D. Plowman, New York, NY (US); Stefan Scherer, Preverenges (CH); Norbert Wild, Geretsried/Gelting (DE)

(73) Assignee: Genentech, Inc., South San Francisco, CA (US)

(21) Appl. No.: 14/289,524

(22) Filed: May 28, 2014

Related U.S. Application Data
(63) Continuation of application No. PCT/EP2012/074184, filed on Dec. 3, 2012.

(30) Foreign Application Priority Data
Dec. 5, 2011 (EP) 11191922.1
Jun. 8, 2012 (EP) 12171293.9

(51) Int. Cl. A61K 39/395; C07K 16/22; G01N 33/574; A61K 31/337

(52) U.S. Cl. CPC A61K 39/39558; A61K 31/337; C07K 16/22; G01N 33/57415; G01N 2333/475
USPC 424/133.1; 424/158.1; 530/389.2; 436/501

(57) ABSTRACT

The present invention provides methods for improving the treatment effect of a chemotherapy regimen of a patient suffering from HER2 positive breast cancer, in particular locally recurrent or metastatic HER2 positive breast cancer, by adding bevacizumab (Avastin®) to a chemotherapy regimen by determining the expression level, in particular the blood plasma expression level, of VEGFA and/or VEGFR2 relative to control levels of patients diagnosed with HER2 positive breast cancer, in particular locally recurrent or metastatic HER2 positive breast cancer. The present invention also provides for methods for assessing the sensitivity or responsiveness of a patient to bevacizumab (Avastin®) in combination with a chemotherapy regimen, by determining the expression level, in particular the blood plasma expression level, of VEGFA and/or VEGFR2 relative to control levels in patients diagnosed with HER2 positive breast cancer, in particular locally recurrent or metastatic HER2 positive breast cancer.

### CROSS-REFERENCE TO RELATED APPLICATIONS

This application is a continuation of International Application No. PCT/EP2012/074184 having an international filing date of Dec. 3, 2012, the entire contents of which are incorporated hereby by reference, and which claims benefit under 35 U.S.C. §119 to European Patent Application Nos. 11191922.1 and 12171293.9 filed on Dec. 5, 2011 and Jun. 8, 2012, respectively.

### SEQUENCE LISTING

The instant application contains a Sequence Listing submitted via EFS-Web and hereby incorporated by reference in its entirety. Said ASCII copy, created on May 28, 2014 is named P4820C1_SequenceListing.txt, and is 22,281 bytes in size.

### FIELD OF THE INVENTION

The present invention is directed to methods for identifying which patients diagnosed with HER2 positive breast cancer will most benefit from treatment with an anti-cancer therapy comprising an anti-VEGF antibody.

### BACKGROUND OF THE INVENTION

Angiogenesis contributes to benign and malignant diseases such as cancer development and, especially in cancer, is necessary for primary tumor growth, invasiveness and metastasis. In order to grow, a tumor must undergo an angiogenic switch. Vascular endothelial growth factor (VEGF) is required to induce this angiogenic switch. VEGF and the genes in the VEGF pathway are considered important mediators of cancer progression. The VEGF gene family includes the VEGF gene, also referred to as VEGFA, homologues to VEGF including, placenta growth factor (P1GF), VEGFB, VEGFC, VEGFD, the VEGF receptors, including VEGFR-1 and VEGFR-2 (also referred to as FLT1 and FLK1/KDR, respectively), the VEGF inducers, including hypoxia-inducible factors HIF1α, HIF2 α, and the oxygen sensors PHD1, PHD2 and PHD3.

The importance of this pathway in cancer cell growth and metastasis has led to the development of anti-angiogenesis agents for use in cancer therapy. These therapies include, among others, bevacizumab, pegaptanib, sunitinib, sorafenib and vatalanib. Despite significantly prolonged survival obtained with angiogenesis inhibitors, such as bevacizumab, patients still succumb to cancer. Further, not all patients respond to angiogenesis inhibitor therapy. The mechanism underlying the non-responsiveness remains unknown. Moreover, angiogenesis inhibitor therapy is associated with side effects, such as gastrointestinal perforation, thrombosis, bleeding, hypertension and proteinuria.

Accordingly, there is a need for methods of determining which patients respond particular well to angiogenesis inhibitor therapy.

Plasma samples obtained from three Avastin trials (AVADO study (BO17708) in HER2 negative metastatic breast cancer, AVITA study (BO17706) in metastatic pancreatic cancer and AVAGAST study (BO20904) in metastatic gastric cancer) were recently analyzed for VEGFA expression levels. The sample median VEGFA concentration was prespecified as a cut point to group patients (high vs. low levels of concentration). A greater magnitude of treatment effect on progression-free survival (PFS), as measured by the hazard ratio (HR) of the treatment arm relative to the control arm, was observed for patients with high VEGF-A levels across these studies. A greater magnitude of treatment effect on overall survival (OS) was also observed for patients with high VEGFA levels in the BO17706 and BO20904 studies. The analyses suggest that plasma VEGF-A is potentially predictive of response to treatment with Avastin in these indications.

Analyses of the BO17708 study in HER2 negative metastatic breast cancer and the BO17706 study in metastatic pancreatic cancer also showed an increased duration of PFS for patients with high VEGFR2 levels in the bevacizumab treatment arm as compared to the control arm.

### SUMMARY OF THE INVENTION

An investigation of the status of biomarkers related to angiogenesis and tumorigenesis revealed that the expression levels of VEGFA and VEGFR2 relative to reference levels determined in the entire biomarker patient population correlated with an improved treatment outcome in HER2 positive breast cancer patients. In particular, patients exhibiting an increased expression level of VEGFA relative to reference levels determined in the entire biomarker patient population, demonstrated a prolonged progression-free survival in response to the addition of bevacizumab to the combination chemotherapy of trastuzumab and docetaxel. Patients exhibiting a higher expression level of VEGFR2 relative to reference levels determined in the entire biomarker patient population, demonstrated a prolonged progression free survival in response to the addition of bevacizumab to the combination chemotherapy of trastuzumab and docetaxel.

The present invention therefore relates to a method of determining whether a patient diagnosed with HER2 positive breast cancer is sensitive to the addition of an anti-VEGF antibody to a chemotherapy regimen, by determining an expression level of VEGFA and/or VEGFR2 in a patient sample and comparing it with reference levels. The present invention also relates to a pharmaceutical composition comprising an anti-VEGF antibody, such as bevacizumab, for the treatment of a patient diagnosed with HER2 positive breast and having an increased expression level of VEGFA and/or VEGFR2 relative to reference levels. The present invention further relates to a method for improving the treatment effect of chemotherapy of a patient diagnosed with HER2 positive breast cancer by adding an anti-VEGF antibody, such as bevacizumab, based on an expression level of VEGFA and/or VEGFR2 in a patient sample.

### DETAILED DESCRIPTION OF THE EMBODIMENTS

### 1. Definitions

The terms “administration” or “administering” as used herein mean the administration of a pharmaceutical composition, such as an angiogenesis inhibitor, to a patient in need of such treatment or medical intervention by any suitable means known in the art. Nonlimiting routes of administration include by oral, intravenous, intraperitoneal, subcutaneous, intramuscular, topical, intradermal, intranasal or intrabronchial administration (for example as effected by inhalation). Particularly preferred in context of this invention is parenteral administration, e.g., intravenous administration.

The term “anti-angiogenesis agent” or “angiogenesis inhibitor” refers to a small molecular weight substance, a polynucleotide, a polypeptide, an isolated protein, a recombinant protein, an antibody, or conjugates or fusion proteins thereof, that inhibits angiogenesis, vasculogenesis, or undesirable vascular permeability, either directly or indirectly. It should be understood that the anti-angiogenesis agent includes those agents that bind and block the angiogenic activity of the angiogenic factor or its receptor. For example, an anti-angiogenesis agent is an antibody or other antagonist to an angiogenic agent as defined throughout the specification or known in the art, e.g., but are not limited to, antibodies to VEGF-A or to the VEGF-A receptor (e.g., KDR receptor or Flt-1 receptor), VEGF-trap, anti-PDGFR inhibitors such as Gleevec™ (Imatinib Mesylate). Anti-angiogensis agents also include native angiogenesis inhibitors, e.g., angiostatin, endostatin, etc. See, e.g., Klagsbrun and D'Amore, Annu. Rev. Physiol., 53:217-39 (1991); Streit and Detmar, Oncogene, 22:3172-3179 (2003) (e.g., Table 3 listing anti-angiogenic therapy in malignant melanoma); Ferrara & Alitalo, Nature Medicine 5:1359-1364 (1999); Tonini et al., Oncogene, 22:6549-6556 (2003) (e.g., Table 2 listing known antiangiogenic factors); and Sato. Int. J. Clin. Oncol., 8:200-206 (2003) (e.g., Table 1 lists anti-angiogenic agents used in clinical trials).

The term “antibody” is herein used in the broadest sense and includes, but is not limited to, monoclonal and polyclonal antibodies, multispecific antibodies (e.g., bispecific antibodies), chimeric antibodies, CDR grafted antibodies, humanized antibodies, camelized antibodies, single chain antibodies and antibody fragments and fragment constructs, e.g., F(ab′)₂ fragments, Fab-fragments, Fv-fragments, single chain Fv-fragments (scFvs), bispecific scFvs, diabodies, single domain antibodies (dAbs) and minibodies.

An “anti-VEGF antibody” is an antibody that binds to VEGF with sufficient affinity and specificity. The antibody selected will normally have a binding affinity for VEGF, for example, the antibody may bind hVEGF with a Kd value of between 100 nM-1 pM. Antibody affinities may be determined by a surface plasmon resonance based assay (such as the BIAcore assay as described in PCT Application Publication No. WO2005/012359); enzyme-linked immunoabsorbent assay (ELISA); and competition assays (e.g. RIA's), for example. In certain embodiments, the anti-VEGF antibody of the invention can be used as a therapeutic agent in targeting and interfering with diseases or conditions wherein the VEGF activity is involved. Also, the antibody may be subjected to other biological activity assays, e.g., in order to evaluate its effectiveness as a therapeutic. Such assays are known in the art and depend on the target antigen and intended use for the antibody. Examples include the HUVEC inhibition assay; tumor cell growth inhibition assays (as described in WO 89/06692, for example); antibody-dependent cellular cytotoxicity (ADCC) and complement-mediated cytotoxicity (CDC) assays (U.S. Pat. No. 5,500,362); and agonistic activity or hematopoiesis assays (see WO 95/27062). An anti-VEGF antibody will usually not bind to other VEGF homologues such as VEGF-B or VEGF-C, nor other growth factors such as P1GF, PDGF or bFGF.

The term “bevacizumab” refers to a recombinant humanized anti-VEGF monoclonal antibody generated according to Presta et al. (1997) Cancer Res. 57:4593-4599, also known as “rhuMAb VEGF” or “AVASTIN®”. It comprises mutated human IgG1 framework regions and antigen-binding complementarity-determining regions from the murine anti-human VEGF monoclonal antibody A.4.6.1 that blocks binding of human VEGF to its receptors. Approximately 93% of the amino acid sequence of bevacizumab, including most of the framework regions, is derived from human IgG1, and about 7% of the sequence is derived from the murine antibody A4.6.1. Bevacizumab binds to the same epitope as the monoclonal anti-VEGF antibody A4.6.1 produced by hybridoma ATCC HB 10709.

The term “cancer” refers to the physiological condition in mammals that is typically characterized by unregulated cell proliferation. Examples of cancer include but are not limited to, carcinoma, lymphoma, blastoma, sarcoma and leukemia. More particular examples of such cancers include squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer (including metastic pancreatic cancer), glioblastoma, cervical cancer, ovarian cancer, liver cancer, bladder cancer, hepatoma, breast cancer (including locally advanced, recurrent or metastatic HER-2 negative breast cancer and locally recurrent or metastatic HER2 positive breast cancer), colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

Examples of “physiological or patholigical angiogenic abnormaliries” include, but are not limited to, high grade glioma, glioblastoma, M. Rendu-Osler, von-Hippel-Lindau diseases, hemangiomas, psoriasis, Kaposi's sarcoma, ocular neovascularisation, rheumatoid arthritis, endometriosis, atherosclerosis, myochardial ischemia, peripheral ischemia, cerebral ischemia and wound healing.

The term “chemotherapeutic agent” or “chemotherapy regimen” includes any active agent that can provide an anticancer therapeutic effect and may be a chemical agent or a biological agent, in particular, that are capable of interfering with cancer or tumor cells. Particular active agents are those that act as anti-neoplastic (chemotoxic or chemostatic) agents which inhibit or prevent the development, maturation or proliferation of malignant cells. Examples of chemotherapeutic agents include alkylating agents such as nitrogen mustards (e.g., mechlorethamine, cyclophosphamide, ifosfamide, melphalan and chlorambucil), nitrosoureas (e.g., carmustine (BCNU), lomustine (CCNU), and semustine (methyl-CCNU)), ethylenimines/methylmelamines (e.g., thriethylenemelamine (TEM), triethylene, thiophosphoramide (thiotepa), hexamethylmelamine (HMM, altretamine)), alkyl sulfonates (e.g., busulfan), and triazines (e.g., dacarbazine (DTIC)); antimetabolites such as folic acid analogs (e.g., methotrexate, trimetrexate), pyrimidine analogs (e.g., 5-fluorouracil, capecitabine, fluorodeoxyuridine, gemcitabine, cytosine arabinoside (AraC, cytarabine), 5-azacytidine, 2,2′-difluorodeoxycytidine), and purine analogs (e.g., 6-mercaptopurine, 6-thioguanine, azathioprine, 2′-deoxycoformycin (pentostatin), erythrohydroxynonyladenine (EHNA), fludarabine phosphate, and 2-chlorodeoxyadenosine (cladribine, 2-CdA)); antimitotic drugs developed from natural products (e.g., paclitaxel, vinca alkaloids (e.g., vinblastine (VLB), vincristine, and vinorelbine), docetaxel, estramustine, and estramustine phosphate), epipodophylotoxins (.e.g., etoposide, teniposide), antibiotics (.e.g, actimomycin D, daunomycin (rubidomycin), daunorubicon, doxorubicin, epirubicin, mitoxantrone, idarubicin, bleomycins, plicamycin (mithramycin), mitomycinC, actinomycin), enzymes (e.g., L-asparaginase), and biological response modifiers (e.g., interferon-alpha, IL-2, G-CSF, GM-CSF); miscellaneous agents including platinum coordination complexes (e.g., cisplatin, carboplatin, oxaliplatin), anthracenediones (e.g., mitoxantrone), substituted urea (i.e., hydroxyurea), methylhydrazine derivatives (e.g., N-methylhydrazine (MIH), procarbazine), adrenocortical suppressants (e.g., mitotane (o,p′-DDD), aminoglutethimide); hormones and antagonists including adrenocorticosteroid antagonists (.e.g, prednisone and equivalents, dexamethasone, aminoglutethimide), progestins (e.g., hydroxyprogesterone caproate, medroxyprogesterone acetate, megestrol acetate), estrogens (e.g., diethylstilbestrol, ethinyl estradiol and equivalents thereof); antiestrogens (e.g., tamoxifen), androgens (e.g., testosterone propionate, fluoxymesterone and equivalents thereof), antiandrogens (e.g., flutamide, gonadotropin-releasing hormone analogs, leuprolide), non-steroidal antiandrogens (e.g., flutamide), epidermal growth factor inhibitors (e.g., erlotinib, lapatinib, gefitinib) antibodies (e.g., trastuzumab), irinotecan and other agents such as leucovorin. For the treatment of locally recurrent or metastatic HER2 positive breast cancer, chemotherapeutic agents for administration with bevacizumab include capecitabine, paclitaxel and docetaxel and combinations thereof (see also the examples herein provided).

The term “docetaxel” is an anti-neoplastic agent that binds to free tubulin and promotes the assembly of tubulin into stable microtubules while simultaneously inhibiting their assembly. This leads to the production of microtubule bundles without normal function and to the stabilization of microtubules, blocking cells in the M-phase of the cell cycle and leading to cell death.

The term “effective amount” refers to an amount of a drug alone or in combination with other drug or treatment regimen effective to treat a disease or disorder in a mammal. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, duration of progression free survival (PFS), the response rates (RR), duration of response, and/or quality of life.

The term “expression level” as used herein refers may also refer to the concentration or amount of marker/indicator proteins of the present invention in a sample.

The term “epitope A4.6.1” refers to the epitope recognized by the anti-VEGF antibody bevacizumab (AVASTIN®) (see Muller Y et al., Structure 15 Sep. 1998, 6:1153-1167). In certain embodiments of the invention, the anti-VEGF antibodies include, but are not limited to, a monoclonal antibody that binds to the same epitope as the monoclonal anti-VEGF antibody A4.6.1 produced by hybridoma ATCC HB 10709; a recombinant humanized anti-VEGF monoclonal antibody generated according to Presta et al. (1997) Cancer Res. 57:4593-4599.

The “epitope 4D5” is the region in the extracellular domain of HER2 to which the antibody 4D5 (ATCC CRL 10463) and trastuzumab bind, as described in WO2009/154651, which is incorporated by reference. This epitope is close to the transmembrane domain of HER2, and within Domain IV of HER2, that being amino acid residues from about 489-630—residue numbering without signal peptide. See Garrett et al Mol Cell. 11: 495-505 (2003), Cho et al Nature 421: 756-760 (2003), Franklin et al Cancer Cell 5:317-328 (2004), and Plowman et al. Proc. Natl. Acad. Sci 90: 1746-1750 (1993). To screen for antibodies which bind essentially to the 4D5 epitope, a routine cross-blocking assay such as that described in Antibodies, A Laboratory Manual, Cold Spring Harbor Laboratory, Ed Harlow and David Lane (1988), can be performed. Alternatively, epitope mapping can be performed to assess whether the antibody binds essentially to the 4D5 epitope of HER2 (e.g. any one or more residues in the region from about residue 529 to, about residue 625, inclusive of the HER2 extracellular domain, residue numbering including signal peptide).

A “HER receptor” is a receptor protein tyrosine kinase which belongs to the HER receptor family and includes EGFR, HER2, HER3 and HER4 receptors. The HER receptor will generally comprise an extracellular domain, which may bind an HER ligand and/or dimerize with another HER receptor molecule; a lipophilic transmembrane domain; a conserved intracellular tyrosine kinase domain; and a carboxyl-terminal signaling domain harboring several tyrosine residues which can be phosphorylated. The HER receptor may be a “native sequence” HER receptor or an “amino acid sequence variant” thereof. In one embodiment, the HER receptor is native sequence human HER receptor. The terms “ErbB1,” “HER1”, “epidermal growth factor receptor” and “EGFR” are used interchangeably herein and refer to EGFR as disclosed, for example, in Carpenter et al, Ann. Rev. Biochem. 56:881-914 (1987), including naturally occurring mutant forms thereof (e.g. a deletion mutant EGFR as in Humphrey et al. PNAS (USA) 87:4207-421 1 (1990)). erbB refers to the gene encoding the EGFR protein product.

The expressions “ErbB2” and “HER2” are used interchangeably herein and refer to the human antigen. HER2 protein described, for example, in Semba et al., PNAS (USA) 82:6497-6501 (1985) and Yamamoto et al. Nature 319:230-234 (1986) (Genebank accession number X03363). The term “erbB2” refers to the gene encoding human ErbB2 and “neu” refers to the gene encoding rat pi 85.

An “anti-Her2 antibody” is an antibody that binds to a HER2 receptor. Optionally, the HER antibody further interferes with HER2 activation or function. In one embodiment, an anti-HER2 antibody of the present invention is an anti-HER2 antibody that binds the 4D5 epitope on HER2 polypeptide, or in another embodiment, trastuzumab.

Humanized HER2 antibodies include huMAb4D5-1, huMAb4D5-2, huMAb4D5-3, huMAb4D5-4, huMAb4D5-5, huMAb4D5-6, huMAb4D5-7 and huMAb4D5-8 or trastuzumab (i.e., HERCEPTIN®) as described in Table 3 of U.S. Pat. No. 5,821,337 expressly incorporated herein by reference. For the purposes herein, “trastuzumab,” HERCEPTN®, and “huMAb4D5-8” refer to an antibody comprising the light and heavy chain amino acid sequences in SEQ ID NOs. 3 and 4, respectively.

A cancer or cancer cell or tumor that is “HER2 positive” is one which has significantly higher levels of a HER receptor protein or gene compared to a noncancerous cell of the same tissue type. Such overexpression may be caused by gene amplification or by increased transcription or translation. HER receptor overexpression or amplification may be determined in a diagnostic or prognostic assay by evaluating increased levels of the HER protein present on the surface of a cell (e.g. via an immunohistochemistry assay; IHC). Alternatively, or additionally, one may measure levels of HER-encoding nucleic acid in the cell, e.g. via fluorescent in situ hybridization (FISH; see WO98/45479 published October, 1998), southern blotting, or polymerase chain reaction (PCR) techniques, such as quantitative real time PCR (qRT-PCR). One may also study HER receptor overexpression or amplification by measuring shed antigen (e.g., HER extracellular domain) in a biological fluid such as serum (see, e.g., U.S. Pat. No. 4,933,294 issued Jun. 12, 1990; WO91/05264 published Apr. 18, 1991; U.S. Pat. No. 5,401,638 issued Mar. 28, 1995; and Sias et al. J. Immunol. Methods 132: 73-80 (1990)). Aside from the above assays, various in vivo assays are available to the skilled practitioner. For example, one may expose cells within the body of the patient to an antibody which is optionally labeled with a detectable label, e.g. a radioactive isotope, and binding of the antibody to cells in the patient can be evaluated, e.g. by external scanning for radioactivity or by analyzing a biopsy taken from a patient previously exposed to the antibody.

The term “metastasis” or “metastatic” refers to the spread of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. Both stimulatory and inhibitory molecular pathways within the tumor cell regulate this behavior, and interactions between the tumor cell and host cells in the distant site are also significant.

The terms “oligonucleotide” and “polynucleotide” are used interchangeably and refer to a molecule comprised of two or more deoxyribonucleotides or ribonucleotides, preferably more than three. Its exact size will depend on many factors, which in turn depend on the ultimate function or use of the oligonucleotide. An oligonucleotide can be derived synthetically or by cloning. Chimeras of deoxyribonucleotides and ribonucleotides may also be in the scope of the present invention.

The term “overall survival (OS)” refers to the length of time during and after treatment the patient survives. As the skilled person will appreciate, a patient's overall survival is improved or enhanced, if the patient belongs to a subgroup of patients that has a statistically significant longer mean survival time as compared to another subgroup of patients.

The term “patient” refers to any single animal, more specifically a mammal (including such non-human animals as, for example, dogs, cats, horses, rabbits, zoo animals, cows, pigs, sheep, and non-human primates) for which treatment is desired. Even more specifically, the patient herein is a human.

The term “a patient suffering from” refers to a patient showing clinical signs in respect to a disease involving physiological and pathological angiogenesis and/or tumorous disease, such as breast cancer, in particular locally recurrent or metastatic HER2 positive breast cancer.

The term “pharmaceutical composition” refers to a sterile preparation that is in such form as to permit the biological activity of the medicament to be effective, and which contains no additional components that are unacceptably toxic to a subject to which the formulation would be administered.

The term “progression-free survival (PFS)” refers to the length of time during and after treatment during which, according to the assessment of the treating physician or investigator, the patient's disease does not become worse, i.e., does not progress. As the skilled person will appreciate, a patient's progression-free survival is improved or enhanced if the patient belongs to a subgroup of patients that has a longer length of time during which the disease does not progress as compared to the average or mean progression free survival time of a control group of similarly situated patients.

The term “polypeptide” relates to a peptide, a protein, an oligopeptide or a polypeptide which encompasses amino acid chains of a given length, wherein the amino acid residues are linked by covalent peptide bonds. However, peptidomimetics of such proteins/polypeptides are also encompassed by the invention wherein amino acid(s) and/or peptide bond(s) have been replaced by functional analogs, e.g., an amino acid residue other than one of the 20 gene-encoded amino acids, e.g., selenocysteine. Peptides, oligopeptides and proteins may be termed polypeptides. The terms polypeptide and protein are used interchangeably herein. The term polypeptide also refers to, and does not exclude, modifications of the polypeptide, e.g., glycosylation, acetylation, phosphorylation and the like. Such modifications are well described in basic texts and in more detailed monographs, as well as in a voluminous research literature. The term polypeptide also refers to and encompasses the term “antibody” as used herein.

The term “responsive to” in the context of the present invention indicates that a subject/patient suffering, suspected to suffer or prone to suffer breast cancer, in particular locally recurrent or metastatic HER2 positive breast cancer, shows a response to a chemotherapy regimen comprising bevacizumab. A skilled person will readily be in a position to determine whether a person treated with bevacizumab according to the methods of the invention shows a response. For example, a response may be reflected by decreased suffering from the breast cancer, in particular locally recurrent or metastatic HER2 positive breast cancer, such as a diminished and/or halted tumor growth, reduction of the size of a tumor, and/or amelioration of one or more symptoms of the cancer. Preferably, the response may be reflected by decreased or diminished indices of the metastatic conversion of the breast cancer such as the prevention of the formation of metastases or a reduction of number or size of metastases (see, e.g., Eisenhauser et al., New response evaluation criteria in solid tumours: Revised RECIST guideline (version 1.1) Eur. J. Cancer 2009 45: 228-247).

The term “reference level” herein refers to a predetermined value. As the skilled artisan will appreciate the reference level is predetermined and set to meet the requirements in terms of e.g. specificity and/or sensitivity. These requirements can vary, e.g. from regulatory body to regulatory body. It may for example be that assay sensitivity or specificity, respectively, has to be set to certain limits, e.g. 80%, 90% or 95%. These requirements may also be defined in terms of positive or negative predictive values. Nonetheless, based on the teaching given in the present invention it will always be possible to arrive at the reference level meeting those requirements. In one embodiment the reference level is determined in healthy individuals. The reference value in one embodiment has been predetermined in the disease entity to which the patient belongs. In certain embodiments the reference level can e.g. be set to any percentage between 25% and 75% of the overall distribution of the values in a disease entity investigated. In other embodiments the reference level can e.g. be set to the median, tertiles or quartiles as determined from the overall distribution of the values in a disease entity investigated. In one embodiment the reference level is set to the median value as determined from the overall distribution of the values in a disease entity investigated.

In certain embodiments, the term “increase” or “above” refers to a level above the reference level or to an overall increase of 5%, 10%, 20%, 25%, 30%, 40%, 50%, 60%, 70%, 80%, 85%, 90%, 95%, 100% or greater, in VEGFA and/or VEGFR2 level detected by the methods described herein, as compared to the VEGFA and/or VEGFR2 level from a reference sample. In certain embodiments, the term increase refers to the increase in VEGFA and/or VEGFR2 wherein, the increase is at least about 1.5-, 1.75-, 2-, 3-, 4-, 5-, 6-, 7-, 8-, 9-, 10-, 15-, 20-, 25-, 30-, 40-, 50-, 60-, 70-, 75-, 80-, 90-, or 100-fold higher as compared to the VEGFA and/or VEGFR2 level e.g. predetermined from a reference sample. In one preferred embodiment the term increased level relates to a value at or above a reference level.

In certain embodiments, the term “decrease” or “below” herein refers to a level below the reference level or to an overall reduction of 5%, 10%, 20%, 25%, 30%, 40%, 50%, 60%, 70%, 80%, 85%, 90%, 95%, 96%, 97%, 98%, 99% or greater, in VEGFA and/or VEGFR2 level detected by the methods described herein, as compared to the VEGFA and/or VEGFR2 level from a reference sample. In certain embodiments, the term decrease refers to the decrease in VEGFA and/or VEGFR2 level, wherein the decreased level is at most about 0.9-, 0.8-, 0.7-, 0.6-, 0.5-, 0.4-, 0.3-, 0.2-, 0.1-, 0.05-, or 0.01-fold the VEGFA and/or VEGFR2 level from the reference sample or lower.

In certain embodiments, the term “at a reference level” refers to a level that is the same as VEGFA and/or VEGFR2 level detected by the methods described herein, from a reference sample.

A “recurrent” cancer is one which has regrown, either at the initial site or at a distant site, after a response to initial therapy.

The term “sensitive to” in the context of the present invention indicates that a subject/patient suffering, suspected to suffer or prone to suffer from breast cancer, in particular locally recurrent or metastatic HER2 positive breast cancer, shows in some way a positive reaction to treatment with bevacizumab in combination with a chemotherapy regimen. The reaction of the patient may be less pronounced when compared to a patient “responsive to” as described hereinabove. For example, the patient may experience less suffering associated with the disease, though no reduction in tumor growth or metastatic indicator may be measured, and/or the reaction of the patient to the bevacizumab in combination with the chemotherapy regimen may be only of a transient nature, i.e., the growth of (a) tumor and/or (a) metastasis(es) may only be temporarily reduced or halted.

The term “survival” refers to the subject remaining alive, and includes progression free survival (PFS) and overall survival (OS). Survival can be estimated by the Kaplan-Meier method, and any differences in survival are computed using the stratified log-rank test.

By “extending survival” or “increasing the likelihood of survival” is meant increasing PFS and/or OS in a treated subject relative to an untreated subject (i.e. relative to a subject not treated with a VEGF antibody), or relative to a control treatment protocol, such as treatment only with the chemotherapeutic agent, such as those use in the standard of care for locally recurrent or metastatic breast cancer, e.g., capecitabine, taxane, anthracycline, paclitaxel, docetaxel, paclitaxel protein-bound particles (e.g., Abraxane®), doxorubicin, epirubicin, 5-fluorouracil, cyclophosphamide, or trastuzumab (e.g., Herceptin®), or combinations thereof. In one embodiment, such standard of care for treating locally recurrent or metastatic breast cancer is a treatment combination comprising trastuzumab and docetaxel. Survival is monitored for at least about one month, about two months, about four months, about six months, about nine months, or at least about 1 year, or at least about 2 years, or at least about 3 years, or at least about 4 years, or at least about 5 years, or at least about 10 years, etc., following the initiation of treatment or following the initial diagnosis.

The term “hazard ratio (HR)” is a statistical definition for rates of events. For the purpose of the invention, hazard ratio is defined as representing the probability of an event in the experimental arm divided by the probability of an event in the control arm at any specific point in time. “Hazard ratio” in progression free survival analysis is a summary of the difference between two progression free survival curves, representing the reduction in the risk of death on treatment compared to control, over a period of follow-up.

As used herein, “therapy” or “treatment” refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

The term “treatment effect” encompasses the terms “overall survival” and “progression-free survival”.

The term “VEGFA” refers to vascular endothelial growth factor protein A, exemplified by SEQ ID NO:5, Swiss Prot Accession Number P15692, Gene ID (NCBI): 7422. The term “VEGFA” encompasses the protein having the amino acid sequence of SEQ ID NO:5 as well as homologues and isoforms thereof. The term “VEGFA” also encompasses the known isoforms, e.g., splice isoforms, of VEGFA, e.g., VEGF₁₁₁, VEGF₁₂₁, VEGF₁₄₅, VEGF₁₆₅, VEGF₁₈₉ and VEGF₂₀₆, as well as variants, homologues and isoforms thereof, including the 110-amino acid human vascular endothelial cell growth factor generated by plasmin cleavage of VEGF₁₆₅ as described in Ferrara Mol. Biol. Cell 21:687 (2010) and Leung et al. Science 246:1306 (1989), and Houck et al. Mol. Endocrin. 5:1806 (1991). In one embodiment of the present invention, “VEGFA” refers to VEGF₁₂₁ and/or VEGF₁₁₀. In one embodiment of the present invention, “VEGFA” refers to VEGF₁₁₁. In the context of the invention, the term “VEGFA” also encompasses variants and/or homologues thereof, as well as fragments of the sequences, provided that the variant proteins (including isoforms), homologous proteins and/or fragments are recognized by one or more VEGFA specific antibodies, such as antibody clone 3C5 and 26503, which are available from Bender RELIATech and R&D Systems, respectively and A4.6.1 as described in Kim et al., Growth Factors 7(1): 53-64 (1992). In the context of the invention, the term “isoform” of VEGF or VEGF-A refers to both splice isoforms and forms generated by enzymatic cleavage (e.g., plasmin).

In one embodiment, “VEGFA” refers to unmodified VEGF. In the context of the present invention “unmodified” VEGF relates to the unmodified amino acid sequence of VEGF, its isoforms and its cleavage products. Unmodified VEGF can e.g. be produced synthetically or preferably recombinantly in prokaryotic expression systems, e.g. in E. coli. Unmodified VEGF does e.g. not carry a posttranslational modification, like a glycosylation. In the context of the invention, the term “unmodified VEGF-A” also encompasses variants and/or homologues thereof, as well as fragments of VEGF-A, provided that the variant proteins (including isoforms), homologous proteins and/or fragments are recognized by an unmodified VEGF-A specific antibodies, such as antibody clone 3C5, which is available from RELIATech GmbH, Wolfenbüttel, Germany.

The term “VEGFR2” refers to vascular endothelial growth factor receptor 2, exemplified by SEQ ID NO:6, Swiss Prot Accession Number P35968, Gene ID (NCBI): 3791. The term “VEGFR2” encompasses the protein having the amino acid sequence of SEQ ID NO:6 as well as homologues and isoforms thereof. In the context of the invention, the term “VEGFR2” also encompasses variants and/or homologues thereof, as well as fragments of the sequences, provided that the variant proteins (including isoforms), homologous proteins and/or fragments are recognized by one or more VEGFR2 specific antibodies, such as antibody clone 89115 and 89109, which are available from R&D Systems.

### 2. Detailed Embodiments

In the present invention, VEGFA and/or VEGFR2 were identified as markers or predictive biomarkers for survival with an anti-angiogenesis therapy. The terms “marker” and “predictive biomarker” can be used interchangeably and refer to expression levels of VEGFA and/or VEGFR2.

Accordingly, the present invention provides a method of determining whether a patient diagnosed with HER2 positive breast cancer is more or less suitably treated by an anti-cancer therapy comprising an anti-VEGF antibody, the method comprising:

(a) determining an expression level of VEGFA and/or VEGFR2 in a sample derived from a patient diagnosed with HER2 positive breast cancer, and

(b) identifying the patient as more or less suitably treated by an anti-cancer therapy comprising an anti-VEGF antibody based on the expression level in accordance with (a), wherein an expression level of VEGFA and/or VEGFR2 at or above a reference level indicates that the patient is more suitably treated with the anti-cancer therapy, or an expression level of VEGFA and/or VEGFR2 below a reference level indicates that the patient is less suitably treated with the anti-cancer therapy. In one embodiment, whether a patient is suitably treated by an anti-cancer therapy is determined in terms of progression-free survival. In one embodiment, the method further comprises treating the patient with the anticancer therapy. In one embodiment, said anti-cancer therapy comprises an anti-VEGF antibody, an anti-Her2 antibody and a taxane.

The present invention further provides a pharmaceutical composition comprising an anti-VEGF antibody for the treatment of a patient diagnosed with HER2 positive breast cancer, wherein the patient has been identified as more suitably treated by an anti-cancer therapy comprising an anti-VEGF antibody by a method comprising:

(a) determining an expression level of VEGFA and/or VEGFR2 in a sample derived from a patient diagnosed with HER2 positive breast cancer, and

(b) identifying the patient as more or less suitably treated by an anti-cancer therapy comprising an anti-VEGF antibody based on the expression level in accordance with (a), wherein an expression level of VEGFA and/or VEGFR2 at or above a reference level indicates that the patient is more suitably treated with the anti-cancer therapy, or an expression level of VEGFA and/or VEGFR2 below a reference level indicates that the patient is less suitably treated with the anti-cancer therapy. In one embodiment, whether a patient is suitably treated by an anti-cancer therapy is determined in terms of progression-free survival. In one embodiment, said anti-cancer therapy comprises an anti-VEGF antibody, an anti-Her2 antibody and a taxane.

The present invention also provides a method of determining whether a patient diagnosed with HER2 positive breast cancer is sensitive to the addition of an anti-VEGF antibody to a chemotherapy regimen, said method comprising:

(a) determining an expression level of VEGFA and/or VEGFR2 in a sample derived from a patient diagnosed with HER2 positive breast cancer, and

(b) identifying the patient as sensitive to the addition of an anti-VEGF antibody to a chemotherapy based on the expression level in accordance with (a), wherein an expression level of VEGFA and/or VEGFR2 at or above a reference level indicates that the patient is sensitive to the addition of an anti-VEGF antibody to a chemotherapy regimen. In one embodiment, a patient is sensitive to the addition of an anti-VEGF antibody to a chemotherapy regimen is determined in terms of progression-free survival. In one embodiment, the method further comprises treating the patient with the anticancer therapy. In one embodiment, said anti-cancer therapy comprises an anti-Her2 antibody and a taxane.

The present invention further provides a pharmaceutical composition comprising an anti-VEGF antibody for the treatment of a patient diagnosed with HER2 positive breast cancer, wherein the patient has been identified as sensitive to the addition of an anti-VEGF antibody to a chemotherapy by a method comprising:

(a) determining an expression level of VEGFA and/or VEGFR2 in a sample derived from a patient diagnosed with HER2 positive breast cancer, and

(b) identifying the patient as sensitive to the addition of an anti-VEGF antibody to a chemotherapy based on the expression level in accordance with (a), wherein an expression level of VEGFA and/or VEGFR2 at or above a reference level indicates that the patient is sensitive to the addition of an anti-VEGF antibody to a chemotherapy regimen. In one embodiment, a patient is sensitive to the addition of an anti-VEGF antibody to a chemotherapy regimen is determined in terms of progression-free survival. In one embodiment, said anti-cancer therapy comprises an anti-Her2 antibody and a taxane.

The present invention also provides a method for improving the treatment effect of a chemotherapy regimen in a patient diagnosed with HER2 positive breast cancer by adding an anti-VEGF antibody to the chemotherapy regimen, the method comprising:

(a) determining an expression level of VEGFA and/or VEGFR2 in a sample derived from a patient diagnosed with HER2 positive breast cancer;

(b) identifying the patient as sensitive to the addition of an anti-VEGF antibody to a chemotherapy based on the expression level in accordance with (a), wherein an expression level of VEGFA and/or VEGFR2 at or above a reference level indicates that the patient is sensitive to the addition of an anti-VEGF antibody to a chemotherapy regimen; and

(c) administering an effective amount of an anti-VEGF antibody in combination with an effective amount of a chemotherapy regimen to the patient identified as sensitive to the addition of an anti-VEGF antibody to a chemotherapy in accordance with (b). In one embodiment, a patient is sensitive to the addition of an anti-VEGF antibody to a chemotherapy regimen is determined in terms of progression-free survival. In one embodiment, said anti-cancer therapy comprises an anti-Her2 antibody and a taxane.

In one embodiment, said patient is diagnosed with locally recurrent or metastatic HER2 positive breast cancer. In one embodiment, said patient received no previous chemotherapeutic or radiation treatment.

In one embodiment, said anti-VEGF antibody binds the A4.6.1 epitope. More specifically, said anti-VEGF antibody is bevacizumab, even more specifically, an anti-VEGF antibody comprising a variable heavy chain (VH) and a variable light chain (VL), wherein said VH has an amino acid sequence of SEQ ID NO:2 and said VL has an amino acid sequence of SEQ ID NO:1.

In one embodiment, said taxane is docetaxel or paclitaxel, more specifically, docetaxel.

In one embodiment, said anti-HER2 antibody binds the 4D5 epitope. More specifically, said anti-HER2 antibody is trastuzumab, even more specifically, an anti-HER2 antibody comprising a variable heavy chain (VH) and a variable light chain (VL), wherein said VH has an amino acid sequence of SEQ ID NO:4 and said VL has an amino acid sequence of SEQ ID NO:3.

In one embodiment, said expression level is a protein expression level.

In one embodiment, said sample is a blood plasma sample, more specifically, an EDTA-blood plasma sample.

In one embodiment, said expression level is an expression level of VEGFA. In one embodiment, an expression level of VEGFA is an expression level of VEGF₁₁₀. In one embodiment, an expression level of VEGFA is an expression level of VEGF₁₂₁. In one embodiment, an expression level of VEGFA is an expression level of VEGF₁₂₁ and VEGF₁₁₀. In one embodiment, an expression level of VEGFA is an expression level of VEGF₁₁₁. In one embodiment, an expression level of VEGFA is an expression level of unmodified VEGF. In one embodiment, said expression level is an expression level of VEGFR2. In one embodiment, said expression level is expression levels of VEGFA and VEGFR2.

In one embodiment, the diagnostic method of the present invention can be performed in vitro.

In the context of the herein described invention, the expression levels, in particular protein expression levels, of VEGFA and/or VEGFR2, may be considered separately, as individual markers, or in groups of two or more, as an expression profile or marker panel. In the context of the herein described invention an expression profile or marker panel wherein the expression profiles of two or more markers may be considered together may also be referred to as a combined expression level. For example, the expression levels of two or more markers may be added together and compared to a similarly determined control combined expression level. Therefore, the methods of the invention encompass determination of an expression profile, including a combined expression level, based on the expression level of one or more of the markers.

In the context of the herein described invention, and in accordance with the appended illustrative example, for consideration of VEGFA or VEGFR2 separately, the following values were used as the corresponding high or low expression value of the marker: High VEGFA (≧129.1 pg/ml), Low VEGFA (<129.1 pg/ml), High VEGFR2 (≧14.1 ng/ml) and Low VEGFR2 (<14.1 ng/ml). These levels were determined as the sample median, as per a prospective analysis plan. Additionally, optimized levels constituting the cut-off value between high and low expression of a particular marker may be determined by varying the cut-off until the subset of patients above and below the cut-off satisfy a relevant statistical optimality criterion. For example, an optimal cut-point may be chosen to maximize the differences in treatment Hazard Ratio between the subset above and below, or to maximize treatment effect in one sub-group, or any other relevant statistical criterion. The skilled person will, however, understand that the expression level of the particular marker and, therefore, what constitutes a high or low expression level may vary by patient and by patient population. Accordingly, the skilled person will understand that when using detection methods other than those described in the appended illustrative example and studying patients and patient populations other than those described in the appended illustrative example, what the skilled person considers a high and/or low expression level for a particular biomarker may vary from the values herein described. Given the methods herein described, the skilled person can determine what constitutes a high and/or low level of expression of a particular biomarker.

As the skilled artisan will appreciate there are many ways to use the measurements of two or more markers in order to improve the diagnostic question under investigation. In a quite simple, but nonetheless often effective approach, a positive result is assumed if a sample is positive for at least one of the markers investigated.

However, a combination of markers may also be evaluated. The values measured for markers of a marker panel (or a combined expression level), e.g. for VEGFA and VEGFR2, may be mathematically combined and the combined value may be correlated to the underlying diagnostic question. Marker values may be combined by any appropriate state of the art mathematical method. Well-known mathematical methods for correlating a marker combination to a disease or to a treatment effect employ methods like, discriminant analysis (DA) (i.e. linear-, quadratic-, regularized-DA), Kernel Methods (i.e. SVM), Nonparametric Methods (i.e. k-Nearest-Neighbor Classifiers), PLS (Partial Least Squares), Tree-Based Methods (i.e. Logic Regression, CART, Random Forest Methods, Boosting/Bagging Methods), Generalized Linear Models (i.e. Logistic Regression), Principal Components based Methods (i.e. SIMCA), Generalized Additive Models, Fuzzy Logic based Methods, Neural Networks and Genetic Algorithms based Methods. The skilled artisan will have no problem selecting an appropriate method to evaluate a marker combination of the present invention. The method used in correlating marker combinations in accordance with the invention herein disclosed with, for example improved overall survival, progression free survival, responsiveness or sensitivity to addition of bevacizumab to chemotherapeutic agents/chemotherapy regimen and/or the prediction of a response to or sensitivity to bevacizumab (in addition to one or more chemotherapeutic agents/chemotherapy regimen) is selected from DA (i.e. Linear-, Quadratic-, Regularized Discriminant Analysis), Kernel Methods (i.e. SVM), Nonparametric Methods (i.e. k-Nearest-Neighbor Classifiers), PLS (Partial Least Squares), Tree-Based Methods (i.e. Logic Regression, CART, Random Forest Methods, Boosting Methods), or Generalized Linear Models (i.e. Logistic Regression). Details relating to these statistical methods are found in the following references: Ruczinski, I., et al, J. of Computational and Graphical Statistics, 12 (2003) 475-511; Friedman, J. H., J. of the American Statistical Association 84 (1989) 165-175; Hastie, Trevor, Tibshirani, Robert, Friedman, Jerome, The Elements of Statistical Learning, Springer Series in Statistics, 2001; Breiman, L., Friedman, J. H., Olshen, R. A., Stone, C. J. (1984) Classification and regression trees, California: Wadsworth; Breiman, L., Random Forests, Machine Learning, 45 (2001) 5-32; Pepe, M. S., The Statistical Evaluation of Medical Tests for Classification and Prediction, Oxford Statistical Science Series, 28 (2003); and Duda, R. O., Hart, P. E., Stork, D. G., Pattern Classification, Wiley Interscience, 2nd Edition (2001).

Accordingly, the invention herein disclosed relates to the use of an optimized multivariate cut-off for the underlying combination of biological markers and to discriminate state A from state B, e.g. patients responsive to or sensitive to the addition of bevacizumab to a chemotherapy regimen from patients that are poor responders to the addition of bevacizumab therapy to a chemotherapy regimen. In this type of analysis the markers are no longer independent but form a marker panel or a combined expression level.

### 3. Detection of Expression Levels of VEGFA/VEGFR2

The expression level of one or more of the markers VEGFA and VEGFR2 may be assessed by any method known in the art suitable for determination of specific protein levels in a patient sample and is preferably determined by an immunoassay method, such as ELISA, employing antibodies specific for one or more of VEGFA and VEGFR2. Such methods are well known and routinely implemented in the art and corresponding commercial antibodies and/or kits are readily available. For example, commercially available antibodies/test kits for VEGFA and VEGFR2 can be obtained from Bender RELIATech and R&D Systems as clone 3C5 and 26503, from R&D systems as clone 89115 and 89109 and from Roche Diagnostics GmbH as clone 2D6D5 and 6A11D2, respectively. Preferably, the expression levels of the marker/indicator proteins of the invention are assessed using the reagents and/or protocol recommendations of the antibody or kit manufacturer. The skilled person will also be aware of further means for determining the expression level of one or more of VEGFA and VEGFR2 by immunoassay methods. Therefore, the expression level of one or more of the markers/indicators of the invention can be routinely and reproducibly determined by a person skilled in the art without undue burden. However, to ensure accurate and reproducible results, the invention also encompasses the testing of patient samples in a specialized laboratory that can ensure the validation of testing procedures.

VEGF₁₂₁ and VEGF₁₁₀ protein can be detected using any method known in the art. For example, tissue or cell samples from mammals can be conveniently assayed for, e.g., proteins using Westerns, ELISAs, etc. Many references are available to provide guidance in applying the above techniques (Kohler et al., Hybridoma Techniques (Cold Spring Harbor Laboratory, New York, 1980); Tijssen, Practice and Theory of Enzyme Immunoassays (Elsevier, Amsterdam, 1985); Campbell, Monoclonal Antibody Technology (Elsevier, Amsterdam, 1984); Hurrell, Monoclonal Hybridoma Antibodies: Techniques and Applications (CRC Press, Boca Raton, Fla., 1982); and Zola, Monoclonal Antibodies: A Manual of Techniques, pp. 147-1 58 (CRC Press, Inc., 1987)).

If reference is made to the detection or level of VEGF₁₂₁ and VEGF₁₁₀ this means that the sum of both molecules is measured, e.g., using an assay that detects both VEGF₁₂₁ and VEGF₁₁₀. Assays that detect both molecules VEGF₁₂₁ and VEGF₁₁₀ include, e.g., assays that have a sensitivity for the corresponding other form, (i.e. for VEGF₁₂₁ if VEGF₁₁₀ is better recognized, or for VEGF₁₁₀ if VEGF₁₂₁ is better recognized, respectively) of at least 25%. In certain embodiments, in the assays have sensitivity to the corresponding other form of at least 50%, 75%, 80%, 85%, 90% or above. In one embodiment both VEGF₁₂₁ and VEGF₁₁₀ are measured with essentially the same sensitivity.

As to detection of VEGF₁₂₁ and VEGF₁₁₀ protein, various assays are available. For example, the sample may be contacted with an antibody or an antibody combination (e.g. in a sandwich assay) preferentially or specifically binding the short VEGF-A isoforms, VEGF₁₂₁ and VEGF₁₁₀, respectively as compared to the longer naturally occurring VEGF-A isoforms VEGF₁₆₅ and VEGF₁₈₉, respectively. Preferably the short isoforms are detected with an at least 3-fold higher sensitivity as compared to the longer isoforms. An at least 3-fold higher sensitivity is acknowledged if a standard curve is established using a short isoform (purity at least 90% by SDS-PAGE and concentration determined by OD 280 nm) and for a long isoform at a predetermined concentration (purity at least 90% by SDS-PAGE and concentration determined by OD 280 nm) using the same reagents and the same standard curve the value read of the standard curves is only one third or less of the expected concentration. Also preferred the sensitivity for the short isoforms is at least 4-fold, 5-fold, 6-fold, 7-fold, 8-fold or 9-fold higher as compared to the long isoforms, especially as compared to VEGF₁₆₅.

In one embodiment both short isoforms VEGF₁₂₁ and VEGF₁₁₀ are specifically detected. Such specific detection is e.g. possible if antibodies, especially monoclonal antibodies are used and employed that bind to the sequence generated by joining exons 4 and 8 in VEGF₁₂₁ or the free C-terminal end of VEGF₁₁₀, respectively. Such VEGF₁₁₀ anti C-terminus antibody does not bind to any VEGF-A isoform comprising amino acid 110 as part of a longer polypeptide chain or to shorter VEGF-A fragments ending e.g. at amino acid 109. The monoclonal antibody that binds to the sequence generated by joining exons 4 and 8, respectively, in VEGF₁₂₁ will not bind to the amino acid sequences comprised in the longer VEGF isoforms 165 and 189, respectively, since therein other amino acid sequences are present due to the joining of exon 4 and exon 7, and of exon 4 and exon 5, respectively (see: Ferrara, N., Mol. Biol. of the Cell 21 (2010) 687-690). Specific binding in the above sense is acknowledged, if the antibody used exhibits less than 10% cross-reactivity with a shorter fragment and less than 10% cross-reactivity with those VEGF-A isoforms not having a free C-terminal amino 110 in case of the anti-VEGF₁₁₀ antibody, or those isoforms not comprising the sequence generated by joining exons 4 and 8 in case of the anti-VEGF₁₂₁ antibody, respectively. Also preferred the cross-reactivity will be less than 5%, 4%, 3%, 2% and 1%, respectively, for both shorter fragments and not having a free C-terminal amino acid 110 or VEGF isoforms not having the sequence generated by joining exons 4 and 8, respectively.

Appropriate specific antibodies only binding the short VEGF isoforms VEGF₁₂₁ or VEGF₁₁₀, respectively, can be obtained according to standard procedures. Usually a peptide representing or comprising the C-terminal most at least 4, 5, 6, 7, 8, 9, 10 or more amino acids of VEGF₁₁₀ or a peptide representing or comprising at least 5, 6, 7, 8, 9, 10 or more amino acids comprising amino acids C-terminal and N-terminal to amino acid 115 of VEGF₁₂₁, respectively, will be synthesized, optionally coupled to a carrier and used for immunization. Specific polyclonal antibodies can be obtained by appropriate immunosorption steps. Monoclonal antibodies can easily be screened for reactivity with VEGF₁₂₁ or VEGF₁₁₀, respectively, and appropriate low cross-reactivity. Low cross-reactivity in terms of the VEGF110-specific antibody can be assessed for both shorter fragments of VEGF₁₁₀ (e.g. lacking the C-terminal amino acid of VEGF₁₁₀) and VEGF-A isoforms not having a free C-terminal amino acid of VEGF₁₁₀. Low cross-reactivity in terms of the VEGF₁₂₁-specific antibody can be assessed using VEGF-isoforms containing the amino acid sequences formed upon joining of exon 4 and exon 7, and of exon 4 and exon 5, respectively.

VEGF₁₁₁ protein or nucleic acids can be detected using any method known in the art. For example, tissue or cell samples from mammals can be conveniently assayed for, e.g., proteins using Westerns, ELISAs, mRNAs or DNAs from a genetic biomarker of interest using Northern, dot-blot, or polymerase chain reaction (PCR) analysis, array hybridization, RNase protection assay, or using DNA SNP chip microarrays, which are commercially available, including DNA microarray snapshots. For example, real-time PCR (RT-PCR) assays such as quantitative PCR assays are well known in the art. In an illustrative embodiment of the invention, a method for detecting mRNA from a genetic biomarker of interest in a biological sample comprises producing cDNA from the sample by reverse transcription using at least one primer; amplifying the cDNA so produced; and detecting the presence of the amplified cDNA. In addition, such methods can include one or more steps that allow one to determine the levels of mRNA in a biological sample (e.g., by simultaneously examining the levels a comparative control mRNA sequence of a “housekeeping” gene such as an actin family member). Optionally, the sequence of the amplified cDNA can be determined.

Many references are available to provide guidance in applying the above techniques (Kohler et al., Hybridoma Techniques (Cold Spring Harbor Laboratory, New York, 1980); Tijssen, Practice and Theory of Enzyme Immunoassays (Elsevier, Amsterdam, 1985); Campbell, Monoclonal Antibody Technology (Elsevier, Amsterdam, 1984); Hurrell, Monoclonal Hybridoma Antibodies: Techniques and Applications (CRC Press, Boca Raton, Fla., 1982); and Zola, Monoclonal Antibodies: A Manual of Techniques, pp. 147-1 58 (CRC Press, Inc., 1987).

As to detection of VEGF₁₁₁ protein, various assays are available For example, the sample may be contacted with an antibody or an antibody combination (e.g. in a sandwich assay) preferentially or specifically binding to VEGF₁₁₁ as compared to the longer naturally occurring VEGF-A isoforms VEGF₁₆₅ and VEGF₁₈₉, respectively. Preferably the short isoform VEGF₁₁₁ is detected with an at least 3-fold higher sensitivity as compared to the longer isoforms. An at least 3-fold higher sensitivity is acknowledged if a standard curve is established using a short isoform (purity at least 90% by SDS-PAGE and concentration determined by OD 280 nm) and for a long isoform at a predetermined concentration (purity at least 90% by SDS-PAGE and concentration determined by OD 280 nm) using the same reagents and the same standard curve the value read of the standard curves is only one third or less of the expected concentration. Also preferred the sensitivity for the short isoforms is at least 4-fold, 5-fold, 6-fold, 7-fold, 8-fold or 9-fold higher as compared to the long isoforms.

In one embodiment isoform VEGF₁₁₁ is specifically detected. Such specific detection is e.g. possible if antibodies, especially monoclonal antibodies are used and employed that bind to the exon junction unique for VEGF₁₁₁. Such antibody does not bind to other VEGF-A isoform or cleavage products thereof not comprising this specific exon junction. Specific binding in the above sense is acknowledged, if the antibody used exhibits less than 10% cross-reactivity with other VEGF-A isoforms, like VEGF₁₂₁ or VEGF₁₆₅, respectively, not having this unique exon junction. Also preferred the cross-reactivity to e.g. VEGF₁₂₁ will be less than 5%, 4%, 3%, 2% and 1%, respectively.

Specificity for VEGF₁₁₁ in one embodiment is assessed by comparing VEGF111 (purity at least 90% by SDS-PAGE and concentration determined by OD 280 nm) and VEGF121 (purity at least 90% by SDS-PAGE and concentration determined by OD 280 nm) using the same reagents. If in this comparison the signal obtained for VEGF₁₂₁ material is only one tens or less of the signal as obtained with the VEGF₁₁₁ material, then cross-reactivity towards VEGF₁₂₁ is less than 10%. As the skilled artisan will appreciate the VEGF₁₂₁ signal is preferably read of at a concentration which yields about 50% of the maximal signal for VEGF₁₁₁.

Appropriate specific antibodies only binding the short VEGF isoform VEGF₁₁₁ can be obtained according to standard procedures. Usually a peptide representing or comprising amino acids C-terminal and N-terminal to amino acid 105 of VEGF₁₁₁ will be synthesized, optionally coupled to a carrier and used for immunization. Preferably such peptide will be at least six amino acids long and comprise at least the amino acids 105 and 106 of VEGF₁₁₁. Also preferred it will comprise at least the amino acids 104, 105, 106 and 107 of VEGF₁₁₁. As the skilled artisan will appreciate longer peptides comprising e.g. 3 or more amino acids N- and C-terminal to the exon junction between amino acids 105 and 106 of VEGF₁₁₁ can also be used to obtain antibodies specifically binding VEGF₁₁₁.

Unmodified VEGF protein can be detected using any appropriate method known in the art. Preferably an antibody will be used having at least the preferential binding properties to unmodified VEGF as compared to modified VEGF as MAB 3C5, which is commercially available from RELIATech GmbH, Wolfenbüttel, Germany. For example, tissue or cell samples from mammals can be conveniently assayed for the unmodified VEGF protein using Westerns, ELISAs, etc. Many references are available to provide guidance in applying the above techniques (Kohler et al., Hybridoma Techniques (Cold Spring Harbor Laboratory, New York, 1980); Tijssen, Practice and Theory of Enzyme Immunoassays (Elsevier, Amsterdam, 1985); Campbell, Monoclonal Antibody Technology (Elsevier, Amsterdam, 1984); Hurrell, Monoclonal Hybridoma Antibodies: Techniques and Applications (CRC Press, Boca Raton, Fla., 1982); and Zola, Monoclonal Antibodies: A Manual of Techniques, pp. 147-1 58 (CRC Press, Inc., 1987)).

If reference is made to the detection or level of unmodified VEGF this means that unmodified VEGF-molecules (isoforms or cleavage products) as e.g. bound by MAB 3C5 are measured.

As to detection of unmodified VEGF protein, various assays are available. For example, the sample may be contacted with an antibody or an antibody combination (e.g. in a sandwich assay) preferentially or specifically binding to unmodified VEGF as compared to modified VEGF, e.g. as naturally occurring in a patient's sample. Preferably unmodified VEGF is detected using an antibody specifically binding to unmodified VEGF, i.e., with an antibody having at least 3-fold higher sensitivity for unmodified VEGF₁₆₅ as compared to modified VEGF₁₆₅. Such at least 3-fold higher sensitivity for unmodified VEGF is assessed by comparing VEGF₁₆₅ recombinantly produced in E. coli (purity at least 90% by SDS-PAGE and concentration determined by OD 280 nm) and VEGF₁₆₅ recombinantly produced in HEK cells (purity at least 90% by SDS-PAGE and concentration determined by OD 280 nm) using the same reagents. If in this comparison the signal obtained for the HEK-produced material is only one third or less of the signal as obtained with the E. coli-derived material, then unmodified VEGF is detected with an at least 3-fold higher sensitivity. As the skilled artisan will appreciate the signal is preferably read of at about 50% of the maximal signal. Preferably in this assessment the assay of example 5 is used. Also preferred the antibody specifically binding to unmodified VEGF (VEGF₁₆₅ ex E. coli) is an antibody that detects unmodified VEGF with and at least 4-fold, 5-fold, 6-fold, 7-fold, 8-fold, 9-fold or 10-fold higher sensitivity as compared to the modified VEGF material (VEGF165 ex HEK cells).

In one embodiment unmodified VEGF is specifically detected using an antibody having at least the same binding preference for unmodified VEGF as compared to modified VEGF as the commercially available MAB 3C5. In one embodiment the relative sensitivity for or preferential binding of an antibody to unmodified VEGF is assessed in a sandwich immuno assay, wherein the antibody to unmodified VEGF is used as a capture antibody and a detection antibody is used that binds to an epitope present on all major VEGF-isoforms or cleavage products. In one embodiment the detection antibody will bind to an epitope outside the epitope for MAB 3C5, i.e., it will not bind to an epitope comprised in a synthetic peptide spanning amino acids 33 to 43 of VEGF. Preferably the detection antibody will bind to an epitope comprised in the amino acids ranging from 1 to 32, form 44 to 105, to the last six amino acids of mature VEGF165, or to a conformational epitope not overlapping with the epitope bound by MAB 3C5. In one embodiment the antibody specifically binding unmodified VEGF165 as compared to modified VEGF has the property to bind to an epitope comprised in a synthetic peptide spanning amino acids 33 to 43 of VEGF.

Appropriate specific antibodies specifically binding unmodified VEGF can be obtained according to standard procedures. Usually an isoform of VEGF produced recombinantly in E. coli or obtained synthetically e.g. by solid phase polypeptide synthesis, or a peptide representing or comprising an epitope of VEGF produced recombinantly in E. coli or obtained synthetically e.g. by solid phase polypeptide synthesis will be used as an immunogen. Monoclonal antibodies can easily be produced according to standard protocols and screened for reactivity with unmodified VEGF and appropriate low cross-reactivity with modified VEGF. One convenient and preferred screening method is based on the use of VEGF₁₆₅ recombinantly produced in E. coli (purity at least 90% by SDS-PAGE and concentration determined by OD 280 nm) and of VEGF₁₆₅ recombinantly produced in HEKcells (purity at least 90% by SDS-PAGE and concentration determined by OD 280 nm), respectively.

The expression level of one or more of VEGFA and VEGFR2 may be assessed in a patient sample that is a biological sample. The patient sample may be a blood sample, blood serum sample or a blood plasma sample. In one embodiment, the sample is EDTA-plasma. In one embodiment, the sample is citrate-plasma. Methods of obtaining blood samples, blood serum samples and blood plasma samples are well known in the art. The patient sample may be obtained from the patient prior to or after neoadjuvant therapy or prior to or after adjuvant therapy.

### 4. Methods of Treatment

In the context of the present invention, bevacizumab is to be administered in addition to or as a co-therapy or co-treatment with one or more chemotherapeutic agents administered as part of standard chemotherapy regimen as known in the art. Examples of agents included in such standard chemotherapy regimens include 5-fluorouracil, leucovorin, irinotecan, gemcitabine, erlotinib, capecitabine, taxanes, such as docetaxel and paclitaxel, interferon alpha, vinorelbine, and platinum-based chemotherapeutic agents, such as, carboplatin, cisplatin and oxaliplatin. As demonstrated in the appended illustrative example, the addition of bevacizumab effected an increase in the progression free survival in the patients and/or patient population defined and selected according to the expression level of one or more of VEGFA and VEGFR2. Thus, bevacizumab may be combined with a chemotherapy regimen, such as docetaxel therapy as demonstrated in the appended illustrative example.

Common modes of administration include parenteral administration as a bolus dose or as an infusion over a set period of time, e.g., administration of the total daily dose over 10 min., 20 min., 30 min., 40 min., 50 min., 60 min., 75 min., 90 min., 105 min., 120 min., 3 hr., 4 hr., 5 hr. or 6 hr. For example, 2.5 mg/kg of body weight to 15 mg/kg of body weight bevacizumab (Avastin®) can be administered every week, every 2 weeks or every 3 weeks, depending on the type of cancer being treated. Examples of dosages include 2.5 mg/kg of body weight, 5 mg/kg of body weight, 7.5 mg/kg of body weight, 10 mg/kg of body weight and 15 mg/kg of body weight given every week, every 2 weeks or every 3 weeks. Further examples of dosages are 5 mg/kg of body weight every 2 weeks, 10 mg/kg every 2 weeks, 7.5 mg/kg of body weight every 3 weeks and 15 mg/kg of body weight every 3 weeks. In the context of the herein described invention, low dose bevacizumab includes, for example, dosages of 2.5 mg/kg of body weight every week, 5 mg/kg of body weight every 2 weeks and 7.5 mg/kg of body weight every 3 weeks. In the context of the herein described invention, high dose bevacizumab includes, for example, dosages of 5 mg/kg of body weight every week, 10 mg/kg of body weight every 2 weeks and 15 mg/kg of body weight every 3 weeks.

The skilled person will recognize that further modes of administration of bevacizumab are encompassed by the invention as determined by the specific patient and chemotherapy regimen, and that the specific mode of administration and therapeutic dosage are best determined by the treating physician according to methods known in the art.

The patients selected according to the methods of the present invention are treated with bevacizumab in combination with a chemotherapy regimen, and may be further treated with one or more additional anti-cancer therapies. In certain aspects, the one or more additional anti-cancer therapy is radiation.

### 5. Kit

The present invention also relates to a diagnostic composition or kit comprising oligonucleotides or polypeptides suitable for the determination of expression levels of one or more of VEGFA and VEGFR2. As detailed herein, oligonucleotides such as DNA, RNA or mixtures of DNA and RNA probes may be of use in detecting mRNA levels of the marker/indicator proteins, while polypeptides may be of use in directly detecting protein levels of the marker/indicator proteins via specific protein-protein interaction. In preferred aspects of the invention, the polypeptides encompassed as probes for the expression levels of one or more of VEGFA and VEGFR2, and included in the kits or diagnostic compositions described herein, are antibodies specific for these proteins, or specific for homologues and/or truncations thereof.

Accordingly, in a further embodiment of the present invention provides a kit useful for carrying out the methods herein described, comprising oligonucleotides or polypeptides capable of determining the expression level of one or more of VEGFA and VEGFR2. The oligonucleotides may comprise primers and/or probes specific for the mRNA encoding one or more of the markers/indicators described herein, and the polypeptides comprise proteins capable of specific interaction with the marker/indicator proteins, e.g., marker/indicator specific antibodies or antibody fragments.

In addition to the methods described above, the invention also encompasses further immunoassay methods for assessing or determining the expression level of one or more of VEGFA and VEGFR2, such as by Western blotting and ELISA-based detection. As is understood in the art, the expression level of the marker/indicator proteins of the invention may also be assessed at the mRNA level by any suitable method known in the art, such as Northern blotting, real time PCR, and RT PCR. Immunoassay- and mRNA-based detection methods and systems are well known in the art and can be deduced from standard textbooks, such as Lottspeich (Bioanalytik, Spektrum Akademisher Verlag, 1998) or Sambrook and Russell (Molecular Cloning: A Laboratory Manual, CSH Press, Cold Spring Harbor, N.Y., U.S.A., 2001). The described methods are of particular use for determining the expression levels of VEGFA and VEGFR2 in a patient or group of patients relative to control levels established in a population diagnosed with breast cancer, in particular locally recurrent or metastatic HER2 positive breast cancer.

The expression level of one or more of VEGFA and VEGFR2, can also be determined on the protein level by taking advantage of immunoagglutination, immunoprecipitation (e.g., immunodiffusion, immunelectrophoresis, immune fixation), western blotting techniques (e.g., (in situ) immuno cytochemistry, affinitychromatography, enzyme immunoassays), and the like. Amounts of purified polypeptide in solution may also be determined by physical methods, e.g. photometry. Methods of quantifying a particular polypeptide in a mixture usually rely on specific binding, e.g., of antibodies. Specific detection and quantitation methods exploiting the specificity of antibodies comprise for example immunoassay methods. For example, concentration/amount of marker/indicator proteins of the present invention in a patient sample may be determined by enzyme linked-immunosorbent assay (ELISA). Alternatively, Western Blot analysis or immunostaining can be performed. Western blotting combines separation of a mixture of proteins by electrophoresis and specific detection with antibodies. Electrophoresis may be multi-dimensional such as 2D electrophoresis. Usually, polypeptides are separated in 2D electrophoresis by their apparent molecular weight along one dimension and by their isoelectric point along the other direction.

As mentioned above, the expression level of the marker/indicator proteins according to the present invention may also be reflected in an increased expression of the corresponding gene(s) encoding the VEGFA and VEGFR2. Therefore, a quantitative assessment of the gene product prior to translation (e.g. spliced, unspliced or partially spliced mRNA) can be performed in order to evaluate the expression of the corresponding gene(s). The person skilled in the art is aware of standard methods to be used in this context or may deduce these methods from standard textbooks (e.g. Sambrook, 2001, loc. cit.). For example, quantitative data on the respective concentration/amounts of mRNA encoding one or more of VEGFA and VEGFR2 can be obtained by Northern Blot, Real Time PCR and the like.

In a further aspect of the invention, the kit of the invention may advantageously be used for carrying out a method of the invention and could be, inter alia, employed in a variety of applications, e.g., in the diagnostic field or as a research tool. The parts of the kit of the invention can be packaged individually in vials or in combination in containers or multicontainer units. Manufacture of the kit follows preferably standard procedures which are known to the person skilled in the art. The kit or diagnostic compositions may be used for detection of the expression level of one or more of VEGFA and VEGFR2 in accordance with the herein-described methods of the invention, employing, for example, immunohistochemical techniques described herein.

For use in the detection methods described herein, the skilled person has the ability to label the polypeptides, for example antibodies, or oligonucleotides encompassed by the present invention. As routinely practiced in the art, hybridization probes for use in detecting mRNA levels and/or antibodies or antibody fragments for use in immunoassay methods can be labelled and visualized according to standard methods known in the art, nonlimiting examples of commonly used systems include the use of radiolabels, enzyme labels, fluorescent tags, biotin-avidin complexes, chemiluminescence, and the like.

The figures show:

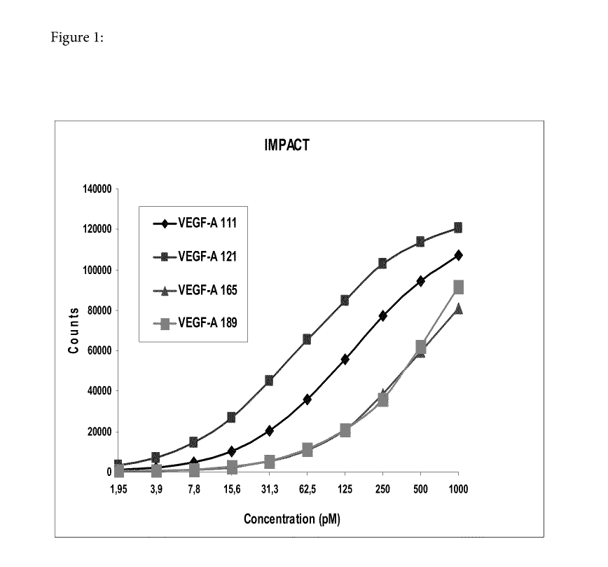

FIG. 1: Measurements of increasing concentrations of VEGF₁₁₁, VEGF₁₂₁, VEGF₁₆₅ and VEGF₁₈₉ as measured on an IMPACT chip.

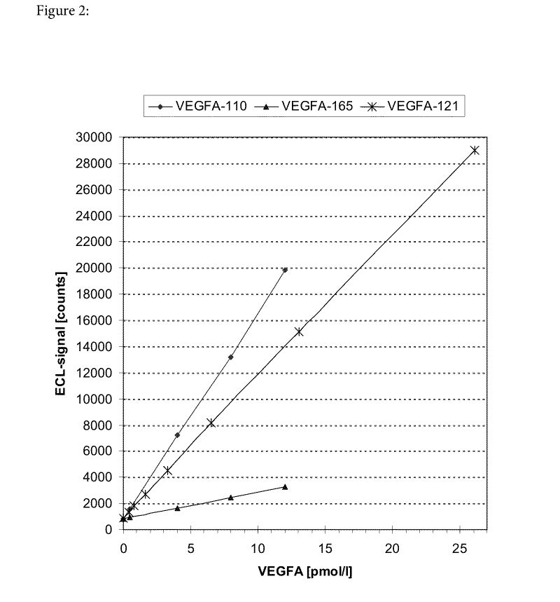

FIG. 2: Measurements of increasing concentrations of VEGF₁₁₀, VEGF₁₂₁, and VEGF₁₆₅ as measured using the Elecsys® Assay on the automated Elecsys® analyzer.

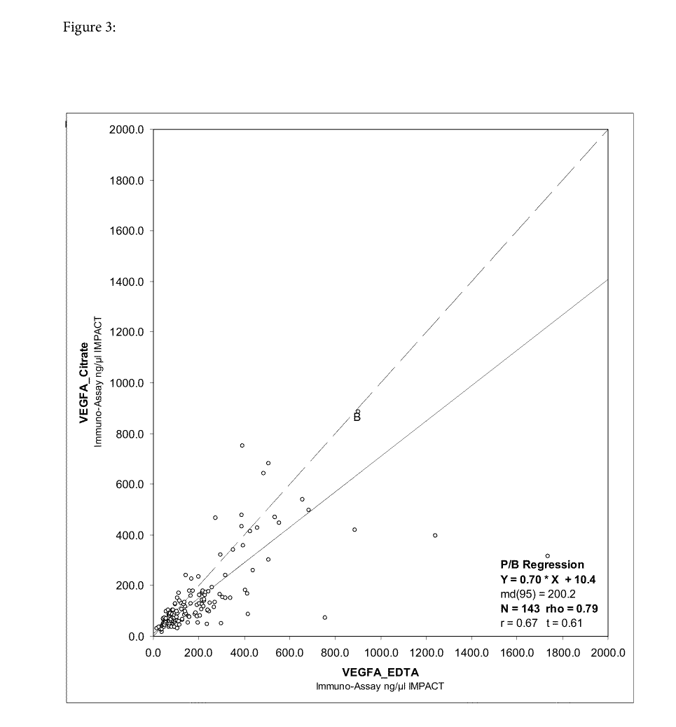

FIG. 3: Data from EDTA- and Citrate samples from the same patients measured twice with the IMPACT assay. The VEGFA concentration is about 40% higher for EDTA-plasma than for Citrate with a Spearman correlation for the EDTA-Citrate method comparison of about 0.8.

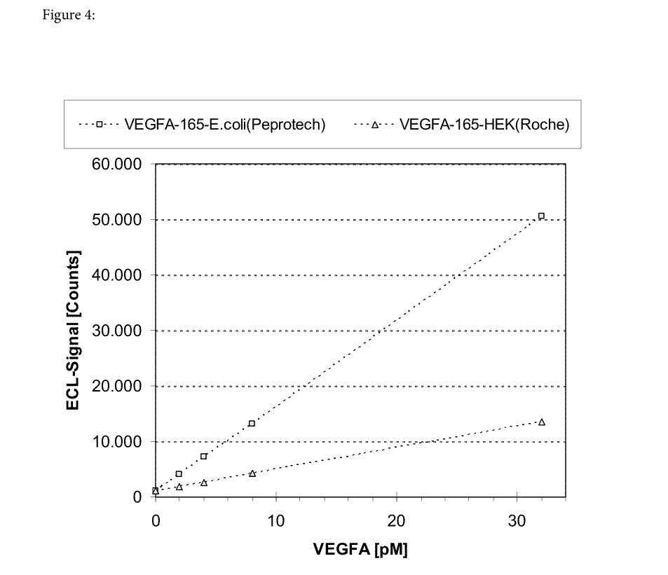

FIG. 4: Shown are the counts (ECL-signal) measured when increasing concentrations of VEGF₁₆₅, produced recombinantly in E. coli or in HEK-cells, respectively, were measured on the automated Elecsys® analyzer.

The present invention is further illustrated by the following non-limiting illustrative example.

### Example 1

### Bevacizumab in Combination with Trastuzumab/Docetaxel Compared with Trastuzumab/Docetaxel Alone as First Line Treatment for Patients with HER2 Positive Locally Recurrent or Metastatic Breast Cancer—AVEREL Study

The primary objective of the clinical trial disclosed herein was to compare Progression Free Survival (PFS) in patients randomized to bevacizumab in combination with trastuzumab/docetaxel versus patients randomized to trastuzumab/docetaxel alone. The secondary objectives were to evaluate Overall Survival (OS); Best Overall Response (OR); Duration of Response (DR); Time to Treatment Failure (TTF); Safety and tolerability of combining bevacizumab with trastuzumab and docetaxel; and finally Quality of Life.

Specifically, the study described herein were to determine (1) that bevacizumab at 15 mg/kg every 3 weeks+trastuzumab at 8 mg/kg loading dose followed by 6 mg/kg every 3 weeks until disease progression+docetaxel 100 mg/m2 every 3 weeks for a minimum of 6 Cycles confers a positive treatment effect on the primary variable of PFS when compared to trastuzumab 8 mg/kg loading dose followed by 6 mg/kg every 3 weeks until disease progression+docetaxel 100 mg/m2 every 3 weeks for a minimum of 6 Cycles; and (2) that bevacizumab at 15 mg/kg every 3 weeks+trastuzumab 8 mg/kg loading dose followed by 6 mg/kg every 3 weeks until disease progression+docetaxel 100 mg/m2 every 3 weeks for a minimum of 6 Cycles has an acceptable safety profile.

### Study Design

The trial was a randomized, open label, 2-arm, multicentre, phase III study. Patients were randomly assigned to treatment groups on a 1:1 basis through a central randomization process. A block design randomization procedure was used. In order to avoid an imbalance of important prognostic factors in the patient population between the two treatment arms, patients were stratified, according to the following criteria:
Prior adjuvant/neo-adjuvant taxane/time to relapse since last dose of adjuvant/neoadjuvant chemotherapy. Patients were initially stratified for prior treatment with taxanes (Yes versus No). If ‘no prior taxanes’, a second stratification was performed i.e. never received adjuvant/neo-adjuvant chemotherapy or relapse ≧12 months since last dose of chemotherapy versus <12 months since last dose of chemotherapy.
Trastuzumab as part of adjuvant treatment versus no trastuzumab;
Hormone receptor (ER/PgR) status (positive versus negative); and
Measurable disease (Yes versus No)

Patients were randomized to one of the following two arms:
Arm A: Trastuzumab 8 mg/kg loading dose followed by 6 mg/kg every 3 weeks until disease progression+Docetaxel 100 mg/m2 every 3 weeks for a minimum of 6 Cycles (or up to disease progression or unacceptable toxicity, whichever occurs first). After 6 Cycles with no progression or toxicity docetaxel may be continued for additional Cycles at the discretion of the investigator.
Arm B: Trastuzumab 8 mg/kg loading dose followed by 6 mg/kg every 3 weeks until disease progression+Docetaxel 100 mg/m2 every 3 weeks for a minimum of 6 Cycles (or up to disease progression or unacceptable toxicity, whichever occurs first). After 6 Cycles with no progression or toxicity docetaxel may be continued for additional Cycles at the discretion of the investigator+Bevacizumab 15 mg/kg every 3 weeks until disease progression.

TABLE 1
Study Overview and Dosing Regimen

| Screening/ | | |
|---|---|---|
| Baseline | Drug Treatment Period | Follow-up |
| Screening Day | Drug Treatment Period Day | Follow-up Day |
| −28 to −1 | Each treatment Cycle is 21 | Clinical assessment |
| | days in length | after last dose |
| | | of study treatment |
| | Trastuzumab loading | At: |
| | dose of 8 mg/kg | −1 month [(Day 28) |
| | was administered | for safety] |
| | on Day 1 of Cycle 1, | after last dose of study |
| | 24 hours prior to the | treatment |
| | first dose of | −3 months after last |
| | bevacizumab and/or | dose of study |
| | docetaxel. Then a dose of 6 | treatment |
| | mg/kg was administered on | Thereafter every |
| | Day 1 of each 3-weekly Cycle | 3 months |
| | until disease progression, | |
| | unacceptable toxicity | |
| | (requiring discontinuation of | |
| | study treatment) or | |
| | withdrawal of patient's | |
| | consent. | |
| | Docetaxel initial dose of 100 | |
| | mg/m2 was administered on | |
| | Day 2 of Cycle 1 and | |
| | thereafter the same dose was | |
| | administered on Day 1 of each | |
| | 3-weekly Cycle for a | |
| | minimum of 6 Cycles. | |
| | Bevacizumab initial dose of | |
| | 15 mg/kg was administered on | |
| | Day 2 of Cycle 1 and | |
| | thereafter the same dose was | |
| | administered on Day 1 of each | |
| | 3-weekly Cycle until | |
| | progression, unacceptable | |
| | toxicity (requiring | |
| | discontinuation of study | |
| | treatment) or withdrawal of | |
| | patient's consent. | |

### Length of Study

424 patients were recruited from 60 centers over approximately 29 months and followed for about 26 months for the primary endpoint (PFS).

### End of Study

This was an event driven trial. The analysis of the primary endpoint was performed when 310 events were confirmed in the 424 patients randomized. An additional analysis of overall survival took place approximately 36 months after randomization of the last patient and the trial ended at this point. End of study occurred at the date of the last visit of the last patient participating in this trial which coincided with the final Overall Survival analysis which took place approximately 36 months after randomization of the last patient.

After this clinical cut-off for the final Overall Survival analysis, patients who benefited from study treatment could continue to receive bevacizumab until disease progression.

Number of Patients/Assignment to Treatment Groups 424 patients were randomized 1:1 into two arms of the trial:
205 HER2 positive patients in the trastuzumab/docetaxel arm (Arm A); and
205 HER2 positive patients in the bevacizumab plus trastuzumab/docetaxel treatment arm (Arm B).

Patients were randomly assigned to treatment groups. Patients received their first dose of study treatment on the day of randomization, but no later than 5 working days after randomization. Under no circumstances were patients who enrolled in this study permitted to be re-randomised to this study and enrolled for a second course of treatment.

### Eligible Patient Enrollment Criteria

Pre- and postmenopausal female and male patients with locally recurrent or metastatic HER2 positive breast cancer (excluding primary tumor-T4d-Inflammatory carcinoma). Patients could have received prior radiotherapy for metastatic breast cancer (MBC) provided that it was completed 3 weeks prior to randomization and no more than 30% of marrow-bearing bone was irradiated. Prior adjuvant radiotherapy was allowed provided it finished at least 6 months before randomization.

Patients who received trastuzumab in the adjuvant setting were allowed to be enrolled, provided that ≧6 months had elapsed since last adjuvant administration of trastuzumab. Patients had to have an adequate Left Ventricular Ejection Function at baseline defined as LVEF not below 50% as measured by either echocardiography or MUGA. Patients who were treated with anthracyclines for adjuvant disease could have been included into the study if the maximum cumulative dose was less/equal to 360 mg/m2 of doxorubicin or 720 mg/m2 of epirubicin.

Patients had to have a histologically or cytologically confirmed HER2 positive, pre- or postmenopausal adenocarcinoma of the breast with measurable or non measurable locally recurrent or metastatic disease. Patients had to have a good performance status (ECOG 0-1), normal liver, renal and bone marrow functions, and be free of other serious diseases, which could affect compliance with the protocol or the interpretation of results. They could not be at increased risk of GI perforation, hypertension, proteinuria and wound healing complications, thromboembolism or haemorrhage. Patients with metastatic CNS disease or spinal cord compression caused by metastasis were not eligible. Patients could not have had another primary tumor within the last 5 years (except for adequately treated CIS of the cervix, squamous carcinoma of the skin or basal cell skin cancer). Pregnant or lactating females were excluded.

Full anticoagulation at study entry was allowed as long as the patient had been on a stable level of anticoagulation for at least two weeks at the time of randomization.

Eligibility Critieria:
1. Patients age ≧18 years;
2. Able to comply with the protocol;
3. ECOG PS of ≦1;
4. Life expectancy of ≧12 weeks;
5. Pre- or postmenopausal patients with histologically or cytologically confirmed breast cancer (adenocarcinoma) with measurable or non-measurable, locally recurrent or metastatic lesions (excluding primary tumor-T4d-Inflammatory carcinoma), who were candidates for chemotherapy. Locally recurrent disease could not be amenable to resection with curative intent. ER/PgR and HER2 status had to have been documented;
6. Patients must have had HER2 protein overexpression (3+) as determined by immunohistochemistry (IHC); or amplification of HER2/c-erbB2 as determined by fluorescent in situ hybridization (FISH) or chromogenic in situ hybridization (CISH), of the primary tumor or a metastasis confirmed by the central laboratory prior to ramdomization. Confirmation of HER2 positivity of the primary tumor by the central laboratory was not required in this trial for the patients who previously participated in Roche or Genentech sponsored trials of adjuvant trastuzumab where HER2 status has been centrally confirmed. (e.g. the HERA, BCIRG006, NSABP B31, or Intergroup/NCCTG/H2061s trials);
7. Patients who received trastuzumab in the adjuvant setting were eligible as long as they did not relapsed within 6 months after the last dose of trastuzumab;
8. Patients who were treated with anthracyclines in adjuvant or neo-adjuvant setting are only eligible if they received their last dose >6 months prior to randomization. The maximum cumulative dose must not have exceeded 360 mg/m² for doxorubicin and 720 mg/m² for epirubicin;
9. Patients who were treated with a taxane are only eligible if they received their last adjuvant or neo-adjuvant chemotherapy >12 months prior to randomization;
10. Baseline Left Ventricular Ejection Fraction (LVEF) not below 50% measured by either echocardiography or MUGA;
11. The use of full-dose oral or parenteral anticoagulants was permitted as long as the patient was on a stable level of anticoagulation for at least two weeks at the time of randomization:
   Patients on heparin treatment that had a baseline aPTT between 1.5-2.5 times ULN or patients value before starting heparin treatment
   Patients on low molecular weight heparins (LMWH) that received daily dose of 1.5
   2 mg/kg (of enoxaparin) or appropriate doses of the correspondent anticoagulant, according to package insert
   Patients on coumarin derivatives that had an INR between 2.0 and 3.0 assessed at baseline in two consecutive measurements 1-4 days apart
   Patients not receiving anticoagulant medication must have had an INR≦1.5 and aPTT≦1.5 times ULN within 7 days prior to randomization.

The Exclusion Criteria:
1. Previous chemotherapy for metastatic or locally recurrent breast cancer. Prior hormonal therapy was allowed but must have been discontinued at least 2 weeks prior randomization.
2. Previous radiotherapy for treatment of metastatic breast cancer was not allowed in case:
   More than 30% of marrow-bearing bone had been irradiated
   The last fraction of radiotherapy had been administered within 3 weeks prior to randomization
   Prior adjuvant radiotherapy for breast cancer was allowed, provided it stopped at least 6 months prior to randomization.
3. Other primary tumor (including primary brain tumors) within the last 5 years prior to randomization, except for adequately treated carcinoma in situ of the cervix, squamous carcinoma of the skin, or adequately controlled limited basal cell skin cancer.
4. Evidence of spinal cord compression or current evidence of CNS metastasis. CT or MRI scan of the brain was mandatory (within 4 weeks prior to randomization) in case of clinical suspicion of brain metastasis.
5. History or evidence upon physical/neurological examination of CNS disease (unrelated to cancer) (unless adequately treated with standard medical therapy) e.g. uncontrolled seizures.
6. Major surgical procedure, open biopsy or significant traumatic injury within 28 days prior to study treatment start, or anticipation of the need for major surgery during the course of the study treatment.
7. Existing peripheral neuropathy >CTC Grade 2 at randomization.
8. Inadequate bone marrow function: ANC <1.5×109/L, Platelet count <100×109/L and Hb <9 g/dL.
9. Inadequate liver function:
   serum (total) bilirubin >ULN
   AST and ALT >2.5×ULN
   AST or ALT >1.5×ULN concurrent with serum alkaline phosphatase levels >2.5×ULN at baseline
10. Inadequate renal function:
   i. Serum Creatinine >2.0 mg/dL or 177 μmol/L
   ii. Urine dipstick for proteinuria ≧2+. Patients with ≧2+proteinuria on dipstick urinalysis at baseline should undergo 24 hours urine collection and must demonstrate ≦1 g of protein/24 hr.
11. Chronic daily treatment with corticosteroids (dose of >10 mg/day methylprednisolone equivalent) (excluding inhaled steroids).
12. Chronic daily treatment with aspirin (>325 mg/day) or clopidogrel (>75 mg/day).
13. Uncontrolled hypertension (systolic >150 mm Hg and/or diastolic >100 mm Hg) or clinically significant (i.e. active) cardiovascular disease: CVA/stroke (≦6 months prior to randomization), myocardial infarction (≦6 months prior to randomization), unstable angina, New York Heart Association (NYHA, Appendix 6) Class 2 or greater Congestive Heart Failure, or serious cardiac arrhythmia requiring medication
14. History or evidence of inherited bleeding diathesis or coagulopathy with the risk of bleeding.
15. History of abdominal fistula, gastrointestinal perforation, or intra-abdominal abscess within 6 months prior to randomization.
16. Active infection requiring i.v. antibiotics at randomization.
17. Serious non-healing wound, peptic ulcer, or bone fracture.
18. Evidence of any other disease, metabolic or psychological dysfunction, physical examination finding, or clinical laboratory finding giving reasonable suspicion of a disease or condition that contraindicates use of an investigational drug, or that may affect patient compliance with study routines, or place the patient at high risk from treatment complications.
19. Pregnant or lactating females. Serum pregnancy test to be assessed within 7 days prior to study treatment start, or within 14 days with a confirmatory urine pregnancy test within 7 days prior to study treatment start.
20. Patients of childbearing potential (women <2 years after last menstruation) not using effective non-hormonal means of contraception (intrauterine contraceptive device, barrier method of contraception in conjunction with spermicidal jelly or surgically sterile).
21. Current or recent (within 30 days prior to starting study treatment) treatment with another investigational drug or participation in another investigational study.
22. Known hypersensitivity to any of the study drugs or excipients.
23. Hypersensitivity to Chinese hamster ovary cell products or other recombinant human or humanized antibodies.

### Results:

The improvement in investigator-assessed PFS, which was not stratified and not censored for non-protocol therapy (NPT), was calculated to be 2.8 months longer than that of the control arm of the study, as described above. Specifically, as summarized in Table 2, the median PFS was 13.7 months for the control group (Arm A) versus 16.5 months for the “bevacizumab” group (Arm B), where the hazard ratio was 0.82 at a 95% CI 0.65,1.02), p-values=0.0775.

TABLE 2
Investigator-Assessed PFS

| | Trast + Doc | Trast + Doc + Bv |
|---|---|---|
| | (n = 208) | (n = 216) |
| No. of subjects with an event | 154 (74.0%) | 153 (70.8%) |
| Progression free survival (months) | | |
| Median | 13.7 | 16.5 |
| (95% CI) | (11.4, 16.3) | (14.1, 19.1) |

| | |
|---|---|
| HR (95% CI) | 0.82 (0.65, 1.02) |
| p-value (log-rank) | 0.0775 |
| Stratified analysis | |
| HR (95% CI) | 0.76 (0.60, 0.96) |
| p-value (log-rank) | 0.0216 |

Further, the independent review committee (IRC) assessed PFS results, which were stratified and censored for NPT, was statistically significant. Specifically, as summarized on Table 3, the median PFS was 13.9 months for the control group (Arm A) versus 16.8 months for the “bevacizumab” group (Arm B), where the hazard ratio was 0.72 at a 95% CI (0.54, 0.94), p-values=0.0162. Overall, the median “Arm B” or “bevacizumab” group's PFS was 2.9 months longer than that of the control arm of the study.

TABLE 3
Independent Review Committee (IRC) PFS

| | Trast + Doc | Trast + Doc + Bv |
|---|---|---|
| | (n = 208) | (n = 216) |
| No. of subjects with an event | 114 (54.8%) | 111 (51.4%) |
| Progression-free survival (months) | | |
| Median | 13.9 | 16.8 |
| (95% CI) | (11.2, 16.7) | (14.1, 19.5) |

| Stratified analysis | |
|---|---|
| HR (95% CI) | 0.72 (0.54, 0.94) |
| p-value (log-rank) | 0.0162 |

The objective response rate (ORR) results of the study, both investigator-assessed results (INV) and independent review committee (IRC) assessed results are shown on Table 4. Specifically for the INV-assessed ORR, the study showed a 69.9% ORR in the control arm versus a 74.3% in the “bevacizumab” or Arm B. The difference between the two is 4.43%, at a 95% CI (−5.2%, 14.0%), with a p-value of 0.3492. For the IRC-assessed ORR, the study showed a 65.9% ORR in the control arm versus a 76.5% in the “bevacizumab” or Arm B. The difference between the two is 10.59%, at a 95% CI (1.0%, 20.2%), with a p-value of 0.0265.

TABLE 4
Objective Response Rate (ORR)

| | Trast + Doc | | Trast + Doc + Bv | |
|---|---|---|---|---|
| | (n = 176) | | (n = 183) | |
| INV | | | | |
| No. of patients with overall response | 123 | (69.9%) | 136 | (74.3%) |
| CR | 10 | (5.7%) | 10 | (5.5%) |
| PR | 113 | (64.2%) | 126 | (68.9%) |
| 95% CI for overall response | (62.5, 76.6) | | (67.4, 80.5) | |
| Difference in overall response rates | 4.43 | | | |
| 95% CI for difference | (−5.2, 14.0) | | | |
| P-value | 0.3492 | | | |
| IRC | | | | |
| No. of patients with overall response | 116 | (65.9%) | 140 | (76.5%) |
| CR | 2 | (1.1%) | 2 | (1.1%) |
| PR | 114 | (64.8%) | 138 | (75.4%) |

| | | |
|---|---|---|
| 95% CI for overall response | (58.4, 72.9) | (69.7, 82.4) |

| | |
|---|---|
| Difference in overall response rates | 10.59 |
| 95% CI for difference | (1.0, 20.2) |
| P-value | 0.0265 |

Interim overall survival (OS) results of the study is summarized in Table 5, which showed that the median survival rate was 38.3 months in the control arm versus 38.5 months in the “bevacizumab” Arm B, with a hazard ratio (HR) of 1.01, at a 95% CI (0.74, 1.38), and a p-value of 0.9543.

TABLE 5
Interim Overall Survival (OS)

| | Trast + Doc | Trast + Doc + Bv |
|---|---|---|
| | (n = 208) | (n = 216) |
| No. of subjects who died | 78 (37.5%) | 81 (37.5%) |
| Duration of overall survival (months) | | |
| Median | 38.3 | 38.5 |
| (95% CI) | (34.3, NR) | (32.1, NR) |

| HR (95% CI) | 1.01 (0.74, 1.38) |
|---|---|
| p-value (log-rank) | 0.9543 |
| Stratified analysis | |
| HR (95% CI) | 0.94 (0.68, 1.30) |
| p-value (log-rank) | 0.7078 |

Further, a preliminary assessment of safety demonstrated that there were no new safety signals shown by patients in this study.

### Example 2

### Exploratory Biomarker Analysis in AVEREL Study

### Patients and Immunochemical Methods

Blood plasma baseline samples were available for analysis from 162 patients in this trial.

### Blood Plasma Analysis

Blood samples for biomarker discovery and validation were collected from consenting patients in study BO20231. Blood samples (approx 20 mL in total) were collected at baseline (after randomization but before the first administration of study medication) and at time of disease progression.

A total of 4.9 mLs of blood were drawn into a S-monovette® (EDTA) tube. They were mixed immediately thereafter by gentle invertion of the tube and were centrifuged within 30 minutes at approximately 1500 g in centrifuge (room temperature for 10 minutes). Immediately hereafter, supernatant plasma was aliquoted in a clear polypropylene 5 mL transfer tube. Thereafter, plasma was aliquoted into 2 plastic storage tubes (approximately 1.25 ml each). Samples were stored in an upright position at −70° C. In some cases, samples were stored at −20° C. for up to one month and then transferred to −70° C.

Samples were used for measurement of levels of Interleukin-8 (I1-8), Inter-Cellular Adhesion Molecule 1 (ICAM-1), VEGFA, VEGF-C, VEGF receptor-1 (VEGFR1), VEGF Receptor 2 (VEGFR2), VEGF receptor-3 (VEGFR3), basic Fibroblast Growth Factor (bFGF), Platelet Derived Growth Factor-C (PDGF-C), and E-SELECTIN using an Immunological MultiParameter Chip Technique (IMPACT) from Roche Diagnostics GmbH.

### IMPACT Multiplex Assay Technology

Roche Professional Diagnostics (Roche Diagnostics GmbH) has developed a multimarker platform under the working name IMPACT (Immunological MultiParameter Chip Technique). This technology was used for the measurement of the protein markers mentioned above in the “blood plasma analysis” section. The technology is based on a small polystyrene chip manufactured by procedures as disclosed in EP 0939319 and EP 1610129. The chip surface was coated with a streptavidin layer, onto which the biotinylated antibodies were then spotted for every assay. For each marker, spots of antibodies were loaded in a vertical line onto the chip. During the assay, the array was probed with specimen samples containing the specific analytes.

The plasma volume required per specimen for measuring all markers on one chip was 20 μl for chip 1 and 8 μL for chip 2 and chip 3 (see below). The sample volume was applied together with incubation buffer (50 mM HEPES pH 7.2, 150 mM NaCl, 0.1% Thesit, 0.5% bovine serum albumin and 0.1% Oxypyrion as a preservative agent) to give a total reaction volume of 40 μl per chip. After incubation for 12 minutes and washing of the chip using a washing buffer (5 mM Tris pH 7.9, 0.01% Thesit and 0.001% Oxypyrion) the digoxigenylated detection antibody mix was added (40 μL of incubation buffer including a mix of the analyte-specific antibodies labeled with Digoxigenin) and was incubated for an additional 6 minutes to bind onto the captured analytes. The second antibody was finally detected after washing with 40 μL of a reagent buffer (62.5 mM TAPS pH 8.7, 1.25 M NaCl, 0.5% bovine serum albumin, 0.063% Tween 20 and 0.1% Oxypyrion) including an anti-digoxigenin antibody conjugate coupled to fluorescent latex. Using this label, 10 individual binding events in a single spot could be detected, resulting in very high sensitivity down to the fmol/L concentration. Chips were transported into the detection unit, and a charge coupled device (CCD) camera generated an image that was transformed into signal intensities using dedicated software. Individual spots were automatically located at predefined positions and quantified by image analysis. For each marker, lines of 10-12 spots were loaded on the chips, and the concentration of the markers was calculated as mean of at least 5 spots from the respective line on the chip. The advantages of the technology are the ability of multiplexing up to 10 parameters in a sandwich or competitive format. The calibrators and patient samples were measured in duplicate. One run was designed to contain a total of 100 determinations, including calibrators and 2 multi-controls as a run control. Since some of the selected analytes react with each other (i.e VEGFA with VEGFR1 or VEGRF2), the analytes were divided on three different chips as follows:

### Chip 1: VEGFA, VEGF-C, PDGF-C

Chip 2: VEGFR1, VEGFR2, VEGFR3, I1-8, bFGF,

### Chip 3: E-selectin, ICAM-1

TABLE 6
Antibodies used for the present assays

| | Capture | Manufac- | Detection | Manufac- |
|---|---|---|---|---|
| Analyte | antibody | turer | antibody | turer |
| VEGFA | <VEGF-A>M- | Bender | <VEGF>M- | R&D |
| | 3C5 | RELIATech | 26503 | Systems |
| VEGFR2 | <VEGF- | R&D | <VEGF- | R&D |
| | R2>M-89115 | Systems | R2>M-89109 | Systems |

### Statistical Analysis

Sample median was used to dichotomize biomarker values as low (below median) or high (at or above median).

Hazard Ratio of treatment effect in sub-group of patients with high or low biomarker levels were estimated with proportional hazard cox regression analysis.

In addition, proportional hazard cox regressions was used to evaluate the association between biomarker level and treatment effect. The model included the following covariates: trial treatment, biomarker level, binary stratification factors (ER/PgR status, measurable disease at baseline, Prior adjuvant/neo-adjuvant taxane/time to relapse since the last dose of adjuvant/neo-adjuvant chemotherapy, prior Trastuzumab neoadjuvant therapy), interaction term of treatment by biomarker level. Wald test for the interaction term was used to determine the association between biomarker level and treatment effect. P-value below 0.05 was considered significant.

### Statistical Methods

### Analysis Populations

A biomarker evaluable population was defined in this study, consisting of all patients who received any component of study medication and had marker levels at baseline for any of the following biomarkers assessed as described above and with commercially available antibodies: VEGF-A, VEGF-C, VEGF-R1, VEGF-R2, E-selectin, VEGFR-3, IL-8, bFGF, PDGF-C, ICAM-1.

### Adjustment for Multiplicity

In order to avoid inflation of the type I error a hierarchy was applied to the biomarkers as shown below. Each biomarker was tested at the two-sided 5% a level, and only if statistical significance was met, the next biomarker in the hierarchy was tested.

1. VEGF-A

2. VEGF-R2

3. ICAM-1

4. bFGF

5. IL-8

6. VEGF-R1

7. PDGF-C

8. VEGF-C

9. VEGFR-3

10. E-selectin

Efficacy Analyses

PFS and OS have been defined as specified in the Data Reporting Analysis Manual (DRAM) for Study BO20231. The analyses of biomarker data has been based on the PFS (Investigator assessed) data at the time of final PFS analysis. The sample median biomarker concentration was used as the cut point to group patients (high vs. low levels of concentration).

### Analyses of Progression Free

The following analyses have been performed for PFS on the biomarker evaluable population:
Median PFS (95% CI) estimated from Kaplan-Meier curves for patients with low and patients with high biomarker levels
Unstratified (& stratified for PFS) HR (95% CI) from the Cox model for patients with low and patients with high biomarker levels
Unstratified HR (95% CI) from Cox model by quartile of biomarker levels
An interaction test for biomarker evaluable patients has been performed to assess whether the biomarker is predictive of treatment benefit on PFS with
bevacizumab for patients with HER2 positive locally recurrent or metastatic breast cancer, using a Cox model that includes baseline biomarker levels (as a binary variable dichotomized as high and low at the sample median), treatment, baseline
prognostic factors and the interaction term (baseline biomarker level by treatment).

### Results

### Blood Plasma Markers

The baseline descriptive statistics of the biomarkers are presented in Table 7.

TABLE 7
Descriptive Statistics of Biomarker Values (Baseline)

| | VEGFA | VEGFR2 |
|---|---|---|
| | (pg/mL) | (ng/mL) |
| min | 15.4 | 6.4 |
| qu 25% | 77.0 | 11.8 |
| median | 129.1 | 14.1 |
| qu 75% | 237.1 | 17.3 |
| max | 1735 | 26.7 |
| mean | 204.8 | 14.8 |
| sd | 214.85 | 3.93 |

Table 8 presents the results of the analysis of the association of VEGFA or VEGFR2 with treatment effect on Investigator assessed progression free survival.

TABLE 8

| | HR (95% CI) |
|---|---|
| VEGFA low | 0.83 (0.50; 1.36) |
| VEGFA high | 0.70 (0.43; 1.14) |
| VEGFR2 low | 0.95 (0.58; 1.55) |
| VEGFR2 high | 0.67 (0.40; 1.10) |

In this analysis, for VEGFA, Low VEGFA (<129.1 pg/ml) and High VEGFA (≧129.1 pg/ml), and for VEGFR2, Low VEGFR2 (<14.1 ng/ml) and High VEGFR2 (≧14.1 ng/ml) were used.

Median PFS in the subgroup of patients with high baseline plasma VEGF-A was 8.5 months with Trast+Doc versus 16.6 months with Trast+Doc+Bv. Corresponding values among patients with low baseline plasma VEGF-A were 13.6 and 16.5 months, respectively.

These results show that the Hazard Ratio for treatment effect is better in the subset of patients with high VEGFA compared to patients with low VEGFA. These results also show that the Hazard Ratio for treatment effect is better in the subset of patients with high VEGFR2 compared to patients with low VEGFR2. The same trend is observed when comparing low and high dose bevacizumab to placebo, the statistical evidence of difference between high and low biomarker sub-group is stronger in the patients treated with low dose bevacizumab. Therefore, VEGFA and VEGFR2 are each independent predictive biomarkers for bevacizumab treatment effect on Progression Free Survival.

### Example 3

### Detection of Shorter Isoforms of VEGF-A Using the IMPACT Assay

This example demonstrates that, based on the antibodies used for detection of VEGF-A on the IMPACT platform, the shorter isoforms of VEGF-A are preferentially measured as compared to the longer isoforms of VEGF-A.

The assay was performed as described above under the section relating to the IMPACT technology using the antibodies listed in the table before the “statistical analysis” section.

Four different VEGF-A forms, i.e. VEGF₁₁₁, VEGF₁₂₁, VEGF₁₆₅ and VEGF₁₈₉ were available and used in the analysis. VEGF₁₁₁, VEGF₁₂₁ (both derived from expression in E. coli), and VEGF₁₆₅ (obtained recombinantly in an insect cell line) was purchased from R&D Systems, Minneapolis, USA and VEGF₁₈₉ was obtained from RELIATech, Wolfenbüttel, Germany. It has turned out later that VEGF₁₈₉ appears to be rather unstable and that the data obtained with that material cannot be relied upon. As shown in FIG. 1 the shorter isoforms having 111 or 121 amino acids, respectively, which had been produced in E. coli and are not secondarily modified, e.g., not glycosylated, are detected better as compared to the longer isoforms with 165 amino acids. VEGF₁₆₅ had been obtained in an insect cell line and is at least partially glycosylated.

The biologically interesting plasmin cleavage product VEGF₁₁₀ was not available for testing when this assay was carried out, but it has to be expected that detection of this isoform will be comparable to what is seen for the VEGF-molecule with 111 amino acids.

### Example 4

### Detection of Short VEGF Isoforms Using the Elecsys® Analyzer

This example describes experiments demonstrating that an assay using the Elecsys® Analyzer and a corresponding assay can be used to detect short VEGF isoforms in human plasma.

The VEGF-A assay was transferred from IMPACT to the automated in-vitro diagnostics system Elecsys® (Roche Diagnostics GmbH, Mannheim). The same capture antibody as in the IMPACT Assay, <hVEGF-A>-m3C5 (RELIATech, Wolfenbüttel) was used, while the capture antibody <hVEGF-A>-m25603 (R&D Systems, Minneapolis) used on the IMPACT system was replaced by <hVEGF-A>-mA4.6.1 (Genentech, South San Francisco).

The immunoassays running on the automated Elecsys® system are immuno assays using electrochemiluminescense (ECLIA) as the signal generating technology. In the present sandwich assay the biotinylated capture antibody binds to streptavidin coated, magnetic microparticles and the ruthenylated detection antibody allows for signal generation. 75 μl of biotinylated <VEGF-A>-m3C5 at 1.5 μg/ml and 75 μl of ruthenylated <VEGF-A>M-A.4.6.1 at 2 μg/ml both in reaction buffer (50 mM Tris (pH 7.4), 2 m M EDTA, 0.1% thesit, 0.2% bovine IgG, 1.0% bovine serum albumin) were incubated for 9 minutes with 20 μl of sample. 30 μl of a microparticle suspension was added after the first 9 minutes of incubation and the whole mixture then incubated for an additional 9 minutes. During these incubation steps an antibody analyte antibody sandwich is formed that is bound to the microparticles. Finally the microparticles were transferred to the detection chamber of the Elecsys system for signal generation and readout.

The cleavage product/isoform preference of the Elecsys® VEGF-A assay was assessed with purified recombinant proteins: VEGF₁₁₀ (produced by plasmin cleavage at Genentech, South San Francisco), VEGF₁₂₁ and VEGF₁₆₅ (both produced in an insect cell line and supplied by R&D Systems, Minneapolis). The preferential binding of short VEGF isoforms that had been seen with the IMPACT® Assay was confirmed in the Elecsys assay. As shown in FIG. 2, in the Elecsys® assay the isoforms VEGF₁₂₁ and the plasmin cleavage product VEGF₁₁₀, respectively, both were detected with an approximately 5-fold higher sensitivity than VEGF₁₆₅.

### Example 5

### Detection of Short VEGF Isoforms in Plasma Collected in Na Citrate and EDTA

Paired plasma samples were collected from patients with HER2+locally recurrent or metastatic breast cancer in both an EDTA monovette (5 mL)- and Citrate Monovette collection tube (5 mL). Within 30 minutes of blood collection, blood tubes were placed into the centrifuge and spun 1500 g at room temperature for 10 minutes, until cells and plasma were separated Immediately after centrifugation, the plasma was carefully transferred into a propylene transfer tube and then aliquotted equally into 2 storage tubes (half volume each approximately 1.25 mL) using a pipette. The levels of VEGF-A in the samples were measured using the IMPACT Assay described above. As shown in FIG. 3, the VEGFA concentration is about 40% higher for plasma samples collected and stored in EDTA compared to plasma samples collected and stored in citrate with a Spearman correlation for the EDTA-Citrate MC of about 0.8 for baseline samples collected prior to treatment.

### Example 6

### Comparative Measurement of Unmodified and Modified VEGF165 on the Elecsys Analyzer

This example describes experiments demonstrating that that the Elecsys® Analyzer and a corresponding assay can be used to detect unmodified VEGF in human plasma.

The VEGF-A assay was transferred from IMPACT to the automated in-vitro diagnostics system Elecsys® (Roche Diagnostics GmbH, Mannheim). The same capture antibody as in the IMPACT assay, <hVEGF-A>-m3C5 (RELIATech GmbH, Wolfenbüttel) was used, while the detection antibody <hVEGF-A>-m25603 (R&D Systems, Minneapolis) used on the IMPACT system was replaced by <hVEGF-A>-mA4.6.1 (Genentech, South San Francisco).

The immunoassays running on the automated Elecsys system are immuno assays using electrochemiluminescense (ECLIA) as the signal generating technology. In the present sandwich assay the biotinylated capture antibody binds to streptavidin coated, magnetic microparticles and the ruthenylated detection antibody allows for signal generation. 75 μl of biotinylated <VEGF-A>-m3C5 at 1.5 μg/ml and 75 μl of ruthenylated <VEGF-A>M-A.4.6.1 at 2 μg/ml both in reaction buffer (50 mM Tris (pH 7.4), 2 m M EDTA, 0.1% thesit, 0.2% bovine IgG, 1.0% bovine serum albumin) were incubated for 9 minutes with 20 μl of sample. 30 μl of a microparticle suspension was added after the first 9 minutes of incubation and the whole mixture then incubated for an additional 9 minutes. During these incubation steps an antibody-analyte-antibody sandwich is formed that is bound to the microparticles. Finally the microparticles were transferred to the detection chamber of the Elecsys system for signal generation and readout.

The preference of the Elecsys VEGF-A assay was assessed with purified recombinant proteins: VEGF₁₆₅ (produced recombinantly in E. coli by Peprotech) and VEGF₁₆₅ (produced recombinantly in HEK-cells at Roche Diagnostics, Germany). The preferential binding of unmodified VEGF₁₆₅ that had been seen with the IMPACT assay was confirmed in the Elecsys assay. As shown in FIG. 4, in the Elecsys assay the unmodified VEGF₁₆₅ was detected with an approximately 5-fold higher sensitivity than modified VEGF₁₆₅.

1. A method of determining whether a patient diagnosed with HER2 positive breast cancer is more or less suitably treated by an anti-cancer therapy comprising an anti-VEGF antibody, the method comprising:
(a) determining an expression level of VEGFA and/or VEGFR2 in a sample derived from a patient diagnosed with HER2 positive breast cancer, and
(b) identifying the patient as more or less suitably treated by an anti-cancer therapy comprising an anti-VEGF antibody based on the expression level in accordance with (a), wherein an expression level of VEGFA and/or VEGFR2 at or above a reference level indicates that the patient is more suitably treated with the anti-cancer therapy, or an expression level of VEGFA and/or VEGFR2 below a reference level indicates that the patient is less suitably treated with the anti-cancer therapy.

2. The method of claim 1, wherein whether a patient is suitably treated by an anti-cancer therapy is determined in terms of progression-free survival.

3. The method of claim 1, wherein the method further comprises treating the patient with the anticancer therapy.

4. The method of claim 1, wherein said anti-cancer therapy comprises an anti-VEGF antibody, an anti-Her2 antibody and a taxane.

5. A method of determining whether a patient diagnosed with HER2 positive breast cancer is sensitive to the addition of an anti-VEGF antibody to a chemotherapy regimen, said method comprising:
(a) determining an expression level of VEGFA and/or VEGFR2 in a sample derived from a patient diagnosed with HER2 positive breast cancer, and
(b) identifying the patient as sensitive to the addition of an anti-VEGF antibody to a chemotherapy regimen based on the expression level in accordance with (a), wherein an expression level of VEGFA and/or VEGFR2 at or above a reference level indicates that the patient is sensitive to the addition of an anti-VEGF antibody to a chemotherapy regimen.

6. The method of claim 5, wherein whether a patient is sensitive to the addition of an anti-VEGF antibody to a chemotherapy regimen is determined in terms of progression-free survival.

7. The method of claim 5, wherein the method further comprises treating the patient with the anticancer therapy.

8. The method of claim 5, wherein said chemotherapy regimen comprises an anti-Her2 antibody and a taxane.

9. The method of claim 1 or 5, wherein said patient is diagnosed with locally recurrent or metastatic HER2 positive breast cancer.

10. The method of claim 1 or 5, wherein said patient received no previous chemotherapeutic or radiation treatment.

11. The method of claim 1 or 5, wherein said anti-VEGF antibody binds the A4.6.1 epitope.

12. The method of claim 1 or 5, wherein said anti-VEGF antibody is bevacizumab.

13. The method of claim 1 or 5, wherein said anti-VEGF antibody comprises a variable heavy chain (VH) and a variable light chain (VL), wherein said VH has an amino acid sequence of SEQ ID NO:2 and said VL has an amino acid sequence of SEQ ID NO:1.

14. The method of claim 4 or 8, wherein said taxane is docetaxel or paclitaxel.

15. The method of claim 4 or 8, wherein said taxane is docetaxel.

16. The method of claim 4 or 8, wherein said anti-HER2 antibody binds the 4D5 epitope.

17. The method of claim 4 or 8, wherein said anti-HER2 antibody is trastuzumab.

18. The method of claim 4 or 8, wherein said anti-HER2 antibody comprises a variable heavy chain (VH) and a variable light chain (VL), wherein said VH has an amino acid sequence of SEQ ID NO:4 and said VL has an amino acid sequence of SEQ ID NO:3.

19. The method of claim 1 or 5, wherein said expression level is a protein expression level.

20. The method of claim 1 or 5, wherein said sample is a blood plasma sample.

21. The method of claim 1 or 5, wherein said expression level is an expression level of VEGFA.

22. The method of claim 1 or 5, wherein said expression level is an expression level of VEGFR2.

23. A pharmaceutical composition comprising an anti-VEGF antibody for the treatment of a patient diagnosed with HER2 positive breast cancer, wherein the patient has been identified as more suitably treated by an anti-cancer therapy comprising an anti-VEGF antibody in accordance with the method of claim 1.

24. A pharmaceutical composition comprising an anti-VEGF antibody for the treatment of a patient diagnosed with HER2 positive breast cancer, wherein the patient has been identified as sensitive to the addition of an anti-VEGF antibody to a chemotherapy in accordance with the method of claim 5.

25. A kit for carrying out the method of claim 1 or 5, comprising a set of compounds for detecting an expression level of VEGFA and/or VEGFR2, the set comprising antibodies capable of specifically binding to VEGFA and/or VEGFR2.

26. A method for improving the treatment effect of a chemotherapy regimen in a patient diagnosed with HER2 positive breast cancer by adding an anti-VEGF antibody to the chemotherapy regimen, the method comprising:
a) determining an expression level of VEGFA and/or VEGFR2 in a sample derived from a patient diagnosed with HER2 positive breast cancer;
b) identifying the patient as sensitive to the addition of an anti-VEGF antibody to a chemotherapy based on the expression level in accordance with (a), wherein an expression level of VEGFA and/or VEGFR2 at or above a reference level indicates that the patient is sensitive to the addition of an anti-VEGF antibody to a chemotherapy regimen; and
c) administering an effective amount of an anti-VEGF antibody in combination with an effective amount of a chemotherapy regimen to the patient identified as sensitive to the addition of an anti-VEGF antibody to a chemotherapy in accordance with (b).

27. The method of claim 26, wherein a patient is sensitive to the addition of an anti-VEGF antibody to a chemotherapy regimen is determined in terms of progression-free survival.

28. The method of claim 26, wherein said anti-cancer therapy comprises an anti-Her2 antibody and a taxane.

29. The method of claim 26, wherein said patient is diagnosed with locally recurrent or metastatic HER2 positive breast cancer.

30. The method of claim 26, wherein said patient received no previous chemotherapeutic or radiation treatment.

31. The method of claim 26, wherein said anti-VEGF antibody binds the A4.6.1 epitope.

32. The method of claim 26, wherein said anti-VEGF antibody is bevacizumab.

33. The method of claim 26, wherein said anti-VEGF antibody comprises a variable heavy chain (VH) and a variable light chain (VL), wherein said VH has an amino acid sequence of SEQ ID NO:2 and said VL has an amino acid sequence of SEQ ID NO:1.

34. The method of claim 28, wherein said taxane is docetaxel or paclitaxel.

35. The method of claim 28, wherein said taxane is docetaxel.

36. The method of claim 28, wherein said anti-HER2 antibody binds the 4D5 epitope.

37. The method of claim 28, wherein said anti-HER2 antibody is trastuzumab.

38. The method of claim 28, wherein said anti-HER2 antibody comprises a variable heavy chain (VH) and a variable light chain (VL), wherein said VH has an amino acid sequence of SEQ ID NO:4 and said VL has an amino acid sequence of SEQ ID NO:3.

39. The method of claim 26, wherein said expression level is a protein expression level.

40. The method of claim 26, wherein said sample is a blood plasma sample.

41. The method of claim 26, wherein said expression level is an expression level of VEGFA.

42. The method of claim 26, wherein said expression level is an expression level of VEGFR2.


SEQUENCE LISTING (6 sequences)

SEQ ID NO: 1 - protein, 108 aa, Artificial Sequence
  sequence is synthesized
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKR

SEQ ID NO: 2 - protein, 123 aa, Artificial Sequence [sequence omitted]

SEQ ID NO: 3 - protein, 214 aa, Artificial Sequence
  sequence is synthesized
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADTEKHKVYACEVTHQGLSSPVTKSFNRGEC

SEQ ID NO: 4 - protein, 449 aa, Artificial Sequence
  sequence is synthesized
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYLTPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 5 - protein, 232 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 6 - protein, 1356 aa, Homo sapiens
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLEIIILVGTAVIAMFFWLLLVIILRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDSGTTLSSPPV